(19) United States
(12) Patent Application Publication
(10) Pub. No.: US 2019/0382814 A1
(43) Pub. Date: Dec. 19, 2019

(54) BIOSYNTHESIS OF CANNABINOID PRODRUGS AND THEIR USE AS THERAPEUTIC AGENTS

(71) Applicant: Teewinot Technologies Limited, Dublin (IE)

(72) Inventors: Richard C. Peet, Washington, DC (US); Malcolm J. Kavarana, Fairfax, VA (US)

(21) Appl. No.: 16/546,086

(22) Filed: Aug. 20, 2019

Related U.S. Application Data
(63) Continuation of application No. 15/488,273, filed on Apr. 14, 2017.
(60) Provisional application No. 62/327,212, filed on Apr. 25, 2016.
(60) Provisional application No. 62/323,296, filed on Apr. 15, 2016.

(51) Int. Cl. C12P 17/06; C12P 13/04; C12M 1/34; C12M 1/40; C07D 311/80; C07C 227/16

(52) U.S. Cl. CPC C12P 17/06; C12P 13/04; C12M 41/26; C12Y 121/03007; C07D 311/80; C07C 227/16; C12M 21/18

(57) ABSTRACT

The present invention provides methods for producing cannabinoid prodrugs. Also described are pharmaceutically acceptable compositions of the prodrugs and a system for the large-scale production of the prodrugs.

### PRIORITY STATEMENT

This application is a continuation of U.S. patent application Ser. No. 15/488,273 filed on Apr. 14, 2017, and it claims the benefit of priority to U.S. Provisional Applications No. 62/323,296, filed Apr. 15, 2016 and No. 62/327,212, filed Apr. 25, 2016, the contents of which applications are incorporated in their entirety in the present application.

### FIELD OF THE INVENTION

The present invention relates to the biosynthesis of pharmaceutically acceptable prodrugs of cannabinoids. Also described is the production and manipulation of enzymes involved in the synthesis of cannabinoids, and the surprising discovery that pH influences the ratio of cannabinoid prodrugs produced using the inventive methods.

### BACKGROUND OF THE INVENTION

Cannabinoids are terpenophenolic compounds found in Cannabis sativa, an annual plant belonging to the Cannabaceae family. The plant contains more than 400 chemicals and approximately 70 cannabinoids. The latter accumulate mainly in the glandular trichomes. The most active of the naturally occurring cannabinoids is tetrahydrocannabinol (THC), which is used for treating a wide range of medical conditions, including glaucoma, AIDS wasting, neuropathic pain, treatment of spasticity associated with multiple sclerosis, fibromyalgia and chemotherapy-induced nausea. THC is also effective in the treatment of allergies, inflammation, infection, epilepsy, depression, migraine, bipolar disorders, anxiety disorder, drug dependency and drug withdrawal syndromes.

Additional active cannabinoids include cannabidiol (CBD), an isomer of THC, which is a potent antioxidant and anti-inflammatory compound known to provide protection against acute and chronic neuro-degeneration. Cannabigerol (CBG), is another cannabinoid found in high concentrations in hemp. CBG is a high affinity α₂-adrenergic receptor agonist and a moderate affinity 5-HT_{1A} receptor antagonist. CBG is a low affinity CB1 receptor antagonist, and has anti-depressant activity.

Cannabichromene (CBC), another phytocannabinoid possesses anti-inflammatory, anti-fungal and anti-viral properties. Phytocannabinoids have been used as therapeutics to treat a variety of diseases and in plants may play a similar role in the plant's defense mechanisms against disease causing agents.

Despite their known beneficial effects, therapeutic use of cannabinoids is hampered by the high costs associated with growing and maintaining plants on a large scale and the difficulty in extracting, isolating and purifying cannabinoids from plant tissues.

There exists a need, therefore, for developing methodologies that allow large-scale production of cannabinoids and cannabinoid prodrugs in quantities required for therapeutic use. The present invention addresses this need.

### SUMMARY

The present invention provides methods for synthesizing prodrugs of cannabinoids. Also described are representative examples of the inventive prodrugs which can be administered to patients in need of cannabinoid based therapy, for example for treating conditions such as glaucoma, chronic pain, AIDS and in the treatment of cancers.

In one embodiment, the present invention provides a method for producing a prodrug of a cannabinoid of Formula II or Formula III:

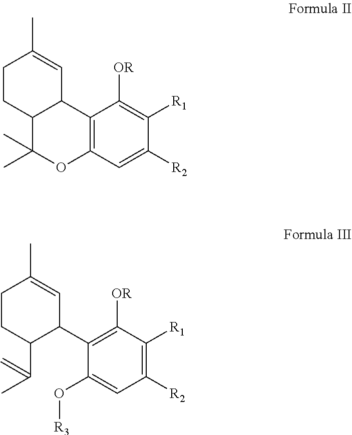

comprising

(a) contacting a compound according to Formula I;

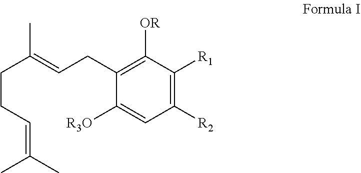

with a cannabinoid synthase to produce a compound according to Formula II or Formula III; and

(b) optionally decarboxylating the Formula II or Formula III compound.

For Formula I, Formula II and Formula III compounds, substituents R and R³ are each independently selected from the group consisting of —H, acetyl, propionyl, 3-hydroxy-2-methylpropionyl, TMS, TBDMS, benzyl, —C(O)[CH₂]ₓ—C(O)OH, —C(O)[CH₂]ₓ—OR⁴, —C(O)[CHR₄]ₓ—C(O)OH, —C(O)[CHR⁴]ₓ—OR⁵, —C(O)[CR⁴R⁵]ₓ—OR⁶, —C(O)O[CH₂]ₓ—OR⁴, —C(O)—CH₂—[OCH₂CH₂]ₓ—OR⁴, —C(O)—C(O)—[OCH₂CH₂]ₓ—OR⁴, —C(O)[CH₂]ₓ—NR⁴R⁵, —C(O)O[CH₂]ₓ—NR⁴R⁵, —C(O)—NH—[CH₂]ₓ—NR⁴R⁵, —C(O)[CH₂]ₓ—N⁺(R⁴)(R⁵))(R⁶)X⁻, —C(O)O[CH₂]ₓ—N⁺(R⁴)(R⁵)(R⁶)X⁻, —C(O)—NH—[CH₂]ₓ—N⁺(R⁴)(R⁵)(R⁶)X⁻—, a L-amino acid residue, a D-amino acid residue, a ß-amino acid residue, a γ-amino acid residue, —P(O)[OY](OZ), and —P(O)[NR⁴NR⁵][OY].

Substituent R¹ in Formula I, Formula II and Formula III is —H, —COOH, —COOR^{a}, or —(CH₂)ₙCOOH, while R² is selected from the group consisting of (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene.

For some Formula II or Formula III compounds substituent R or R³ is —C(O)[CHR₄]ₓ—C(O)OH, —C(O)[CHR⁴]ₓ—OR⁵, —C(O)[CR⁴R⁵]ₓ—OR⁶, —C(O)O[CH₂]ₓ—OR⁴, —C(O)—CH₂—[OCH₂CH₂]ₓ—OR⁴, or —C(O)—C(O)—[OCH₂CH₂]ₓ—OR⁴. For such compounds, substituents R⁴ and R⁵ each independently are —NH₂, —NH(CH₃), —NH(CH₂CH₃), or N(CH₃)₂. For certain other Formula II or III compounds, substituents R⁴ and R⁵ are each independently —H or a (C₁-C₅)alkyl, for example, methyl, ethyl propyl, butyl or t-butyl.

Substituents R⁴, R⁵, and R⁶ are each independently selected from the group consisting of —H, —OH, formyl, acetyl, pivaloyl, and (C₁-C₅)alkyl. In one embodiment R⁴ and R⁵, are each independently —H or a (C₁-C₅)alkyl and the group —NR⁴R⁵ is —NH₂, —NH(CH₃), —NH(CH₂CH₃), or N(CH₃)₂. According to another embodiment, either R⁴ and R⁵ is formyl or acetyl and the group —NR⁴R⁵ is —NH[C(O)H], and —NH[C(O)CH₃]. Substituent R^{a} is a (C₁-C₁₀)alkyl, for example, methyl, ethyl or t-butyl for Formula I, II and III compounds.

For some Formula I, Formula II and Formula III compounds variable “X” is a counter ion derived from a pharmaceutically acceptable acid while variables “Y” and “Z” are each independently selected from the group consisting of —H, (C₁-C₅)alkyl, alkali metal cations, alkaline earth metal cations, ammonium cation, methyl ammonium cation, and pharmaceutically acceptable bases. For compounds in accordance with the invention, subscripts “x” and “n” are selected from the group consisting of 0, 1, 2, 3, 4, 5, and 6.

In one embodiment, for compounds in accordance with the invention, substituent R is selected from the group consisting of —C(O)[CH₂]ₓ—C(O)OH, —C(O)[CH₂]ₓ—OR⁴, —C(O)[CH₂]ₓ—NR⁴R⁵, and —C(O)—CH₂—[OCH₂CH₂]ₓ—OR⁴, substituent R¹ is —COOH, and R² is (C₁-C₁₀)alkyl, for example, a propyl or a pentyl group.

For certain Formula I, Formula II and Formula III compounds R is —C(O)[CH₂]ₓ—OR⁴, subscript “x” is 1, 2, 3, or 4, and R⁴ is —H, or (C₁-C₅)alkyl.

In one embodiment, R is —C(O)—CH₂—[OCH₂CH₂]ₓ—OR⁴, subscript “x” is 1, 2, 3, or 4 and substituent R⁴ is methyl.

According to another embodiment, substituent R is —C(O)[CH₂]ₓ—NR⁴R⁵, subscript “x” is 1, 2, 3, or 4 and substituent groups R⁴ and R⁵ are each independently —H, or (C₁-C₅)alkyl, for example methyl or ethyl.

The present invention also provides a cannabinoid prodrug according to Formula IV or Formula V.

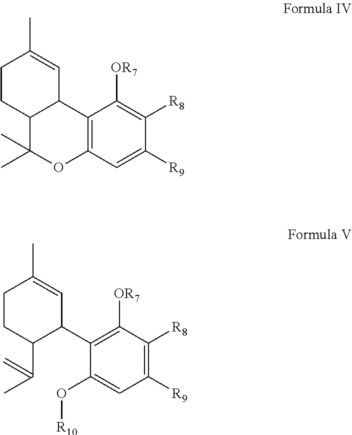

For Formula IV and Formula V compounds R⁷ and R¹⁰ are each independently selected from the group consisting of —H, acetyl, propionyl, 3-hydroxy-2-methylpropionyl, tetrahydropyranyl, —C(O)[CH₂]ₓ—C(O)OH, —C(O)[CH₂]ₓ—OR¹¹, —C(O)[CHR¹¹]ₓ—C(O)OH, —C(O)[CHR¹¹]ₓ—OR¹², —C(O)[CR¹¹R¹²]ₓ—OR¹³, —C(O)O[CH₂]ₓ—OR¹¹, —C(O)—CH₂—[OCH₂CH₂]ₓ—OR¹¹, —C(O)—C(O)—[OCH₂CH₂]ₓ—OR¹¹, —C(O)[CH₂]ₓ—NR¹¹R¹², —C(O)O[CH₂]ₓ—NR¹¹R¹², —C(O)—NH—[CH₂]ₓ—NR¹¹R¹², —C(O)[CH₂]ₓ—N+(R¹¹)(R¹²))(R¹³)X⁻—, —C(O)O[CH₂]ₓ—N⁺(R¹¹)(R¹²))(R¹³)X⁻, —C(O)—NH—[CH₂]ₓ—N⁺(R¹¹)(R¹²))(R¹³)X⁻—, a L-amino acid residue, a D-amino acid residue, a ß-amino acid residue, a γ-amino acid residue, —P(O)[OY](OZ), and —P(O)[NR¹¹NR²][OY].

R⁸ in Formula IV and Formula V is —H, —COOH, —COOR¹¹, or —(CH₂)ₙCOOH, and substituent R^{a} is (C₁-C₁₀)alkyl, for example, methyl, ethyl, or t-butyl and substituent R⁹ is selected from the group consisting of (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene.

In one embodiment, R⁷ and R¹⁰ are each independently —C(O)[CH₂]ₓ—OR¹¹, —C(O)[CHR¹¹]ₓ—C(O)OH, —C(O)[CHR¹¹]ₓ—OR¹², —C(O)[CR¹¹R¹²]ₓ—OR¹³, —C(O)O[CH₂]ₓ—OR¹¹, —C(O)—CH₂—[OCH₂CH₂]ₓ—OR¹¹, and —C(O)—C(O)—[OCH₂CH₂]ₓ—OR¹¹. For such compounds, substituents R¹¹, R¹² and R¹³ are each independently —H or a (C₁-C₅)alkyl, for example, methyl, ethyl, propyl, butyl or t-butyl. For certain other compounds, substituents R¹¹ and R¹² are selected from —NH₂, —NH(CH₃), —NH(CH₂CH₃), or N(CH₃)₂.

For compounds in accordance with Formula IV and V, substituents R¹¹, R¹² and R¹³ are each independently selected from the group consisting of —H, —OH, formyl, acetyl, pivaloyl, and (C₁-C₅)alkyl. In one embodiment R¹¹ and R¹² are —H or a (C₁-C₅)alkyl and the group —NR¹¹R¹² is —NH₂, —NH(CH₃), —NH(CH₂CH₃), or N(CH₃)₂. According to another embodiment, either R¹¹ or R¹² is formyl or acetyl and the group —NR¹¹R¹² is —NH[C(O)H], or —NH[C(O)CH₃]. When R⁸ is —COOR^{a}, substituent R^{a} is (C₁-C₁₀)alkyl, for example, methyl, ethyl or t-butyl.

Variable “X” is a counter ion derived from a pharmaceutically acceptable acid, while variables “Y” and “Z” are each independently selected from the group consisting of —H, (C₁-C₅)alkyl, alkali metal cations, alkaline earth metal cations, ammonium cation, methyl ammonium cation, and pharmaceutically acceptable bases.

For Formula IV and Formula V compounds, subscripts “x” and “n” are independently selected from the group consisting of 0, 1, 2, 3, 4, 5, and 6.

In one embodiment, R⁷ is selected from the group consisting of —C(O)[CH₂]ₓ—C(O)OH, —C(O)[CH₂]ₓ—OR¹¹, —C(O)[CH₂]ₓ—NR¹¹R¹², —C(O)—CH₂—[OCH₂CH₂]ₓ—OR¹¹, and —C(O)[CH₂]ₓ—N⁺(R¹¹)(R¹²)(R¹³)X⁻, substituent R⁸ is —H or —COOH, and R⁹ is propyl, butyl, or pentyl. According to this embodiment, for certain Formula IV and V compounds R⁸ is —H and R⁹ is propyl, or pentyl.

In one embodiment the prodrug moiety at R⁷ is acetyl. According to another embodiment, R⁷ is a pivaloyl moiety.

For certain Formula V compounds, both R⁷ and R¹⁰ are acetyl or pivaloyl, while for some other Formula V compounds R⁷ is —H and R¹⁰ is acetyl or pivaloyl.

For certain inventive compounds, the prodrug moiety at R⁷ is a —C(O)[CH₂]ₓ—OH group or a —C(O)[CH₂]ₓ—OMe group with subscript “x” being 1 or 2. In one embodiment, prodrugs according Formula V are provided where both R⁷ and R¹⁰ are a —C(O)[CH₂]ₓ—OH group or a —C(O)[CH₂]ₓ—OMe group. According to yet another embodiment, R⁷ is —H and R¹⁰ is a —C(O)[CH₂]ₓ—OH or a —C(O)[CH₂]ₓ—OMe group.

In one embodiment, the prodrug moiety at R⁷ is a —C(O)[CH₂]ₓ—N⁺(R¹¹)(R¹²))(R¹³)X⁻ moiety, for example, a —C(O)O[CH₂]—N⁺(CH₃)(CH₂CH₃)₂X⁻, —C(O)O[CH₂]—N⁺(CH₃)₃X⁻, —C(O)O[CH₂]—N⁺(CH₂CH₃)₃X⁻, —C(O)O[CH₂]₂—N⁺(CH₃)₃X⁻, —C(O)O[CH₂]₃—N⁺(CH₃)₃X⁻, or —C(O)O[CH₂]₄—N⁺(CH₃)₃X⁻ group.

For certain Formula IV and V compounds, the prodrug moiety at R⁷ is —C(O)O[CH₂]₄—NH₂, —C(O)O[CH₂]—NH₂, —C(O)O[CH₂]—NH(CH₃), —C(O)O[CH₂]—NH(formyl), or —C(O)O[CH₂]—N(CH₃)₂.

In one embodiment, the prodrug moiety at R⁷ is a polyethylene glycol group, such as a —C(O)—CH₂—[OCH₂CH₂]ₓ—OH or a —C(O)—CH₂—[OCH₂CH₂]ₓ—OCH₃ group, with subscript “x” being 1, 2, 3, or 4. Illustrative of such prodrugs without limitation are —C(O)—CH₂—[OCH₂CH₂]₃—OCH₃, and —C(O)—CH₂—[OCH₂CH₂]₂—OCH₃ groups.

As described above, encompassed within the scope of the invention are cannabinoid prodrugs according to Formula V where R⁷ and R⁰ are both prodrug moieties or only one of R⁷ or R⁰ is a prodrug moiety selected from the group consisting of —C(O)[CH₂]ₓ—N⁺(R¹¹)(R¹²)(R¹³)X⁻ moiety, for example, a —C(O)O[CH₂]—N⁺(CH₃)(CH₂CH₃)₂X⁻, —C(O)O[CH₂]—N⁺(CH₃)₃X⁻, —C(O)O[CH₂]—N⁺(CH₂CH₃)₃X⁻, —C(O)O[CH₂]₄—N⁺(CH₃)₃X⁻, —C(O)O[CH₂]₄—NH₂, —C(O)O[CH₂]—NH₂, —C(O)O[CH₂]—NH(CH₃), —C(O)O[CH₂]—NH(formyl), or —C(O)O[CH₂]—N(CH₃)₂, —C(O)—CH₂—[OCH₂CH₂]ₓ—OH or a —C(O)—CH₂—[OCH₂CH₂]ₓ—OCH₃ group. Illustrative of such prodrugs without limitation are —C(O)—CH₂—[OCH₂CH₂]₃—OCH₃ and —C(O)—CH₂—[OCH₂CH₂]₂—OCH₃.

Also encompassed within the scope of the present invention is a system for producing cannabinoid prodrugs, for example, prodrugs according to Formula VII and VIII respectively.

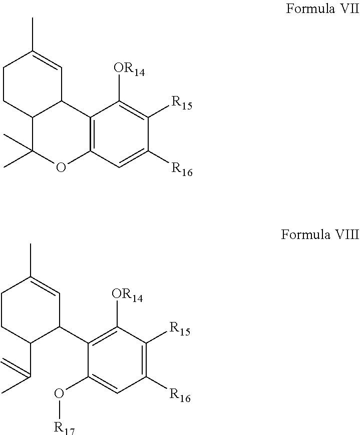

According to the invention, the system for synthesizing Formula VII and VIII compounds comprises: (i) a bioreactor containing a reactant according to Formula VI, a solvent, and a cannabinoid synthase; and

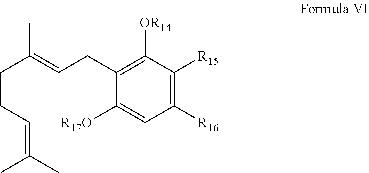

(ii) a control mechanism configured to control at least one condition of the bioreactor, wherein the compound according to Formula VI interacts with the cannabinoid synthase to produce a compound according to Formula VII or Formula VIII.

In one embodiment, the Formula VII and VIII compounds produced using the inventive system are de-carboxylated prior to their use as pharmaceutical or nutraceutical agents.

Substituents R¹⁴ and R¹⁷ in Formula VI, VII, or VIII are each independently selected from the group consisting of —H, acetyl, propionyl, 3-hydroxy-2-methylpropionyl, TMS, TBDMS, benzyl, tetrahydropyranyl, —C(O)[CH₂]ₓ—C(O)OH, —C(O)[CH₂]ₓ—OR¹⁸, —C(O)[CHR¹⁸]ₓ—C(O)OH, —C(O)[CHR¹⁸]ₓ—OR¹⁹, —C(O)[CR¹⁸R¹⁹]ₓ—OR²⁰, —C(O)O[CH₂]ₓ—OR¹⁸, —C(O)—CH₂—[OCH₂CH₂]ₓ—OR¹⁸, —C(O)—C(O)—[OCH₂CH₂]ₓ—OR¹⁸, —C(O)[CH₂]ₓ—NR¹⁸R¹⁹, —C(O)O[CH₂]ₓ—NR¹⁸R¹⁹, —C(O)—NH—[CH₂]ₓ—NR¹⁸R¹⁹, —C(O)[CH₂]ₓ—N⁺(R¹⁸)(R¹⁹))(R²⁰)X⁻, —C(O)O[CH₂]ₓ—N⁺(R¹⁸)(R¹⁹))(R²⁰)X⁻, —C(O)—NH—[CH₂]₁—N⁺(R¹⁸)(R¹⁹))(R²⁰)X⁻, a L-amino acid residue, a D-amino acid residue, a ß-amino acid residue, a γ-amino acid residue, —P(O)[OY](OZ), and —P(O)[NR¹⁸NR¹⁹][OY](OZ).

Substituent R¹⁵ is —H, —COOH, —COOR^{a}, or —(CH₂)ₙCOOH and R¹⁶ is selected from the group consisting of (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene.

For compounds according to Formula VI, VII, or VIII, R^{a} is (C₁-C₁₀)alkyl, for example, methyl, ethyl or butyl and substituents R¹⁸, R¹⁹, and R²⁰ are each independently selected from the group consisting of —H, —OH, formyl, acetyl, pivaloyl, and (C₁-C₅)alkyl.

For some Formula VI, VII, or VIII compounds, R¹⁴ and R¹⁷ are each independently —C(O)[CH₂]ₓ—OR¹⁸, —C(O)[CHR¹⁸]ₓ—C(O)OH, —C(O)[CHR¹⁸]ₓ—OR¹⁹, —C(O)[CR¹⁸R¹⁹]ₓ—OR²⁰, —C(O)O[CH₂]ₓ—OR¹⁸, —C(O)—CH₂—[OCH₂CH₂]ₓ—OR¹⁸, and —C(O)—C(O)—[OCH₂CH₂]ₓ—OR¹⁸. For such compounds, substituents R¹⁸, R¹⁹ and R²⁰ are each independently —H or a (C₁-C₅)alkyl, for example, methyl, ethyl propyl, butyl or t-butyl. For certain other compounds, substituents R¹⁸ and R¹⁹ are selected from —NH₂, —NH(CH₃), —NH(CH₂CH₃), or N(CH₃)₂.

In one embodiment R¹⁸ and R¹⁹, are each independently —H or a (C₁-C₅)alkyl and the group —NR¹⁸R¹⁹ is —NH₂, —NH(CH₃), —NH(CH₂CH₃), and N(CH₃)₂. According to another embodiment R¹⁸ and R¹⁹, are each independently formyl or acetyl and the group —NR¹⁸R¹⁹ is —NH[C(O)H], or —NH[C(O)CH₃].

Variable “X” is a counter ion derived from a pharmaceutically acceptable acid and variables “Y” and “Z” are each independently selected from the group consisting of —H, (C₁-C₅)alkyl, alkali metal cations, alkaline earth metal cations, ammonium cation, methyl ammonium cation, and pharmaceutically acceptable bases. For Formula VI, VII and VIII compounds, subscripts “x” and “n” are independently selected from the group consisting of 0, 1, 2, 3, 4, 5, and 6.

In one embodiment the cannabinoid synthase is a natural enzyme or a recombinant enzyme selected from the group consisting of tetrahydrocannabinolic acid synthase (THCA synthase), tetrahydrocannabivarin acid synthase (THCVA synthase), cannabidiolic acid synthase (CBDA synthase), and cannabichromene acid synthase (CBCA synthase).

The foregoing general description and the detailed description to follow are exemplary and explanatory and are intended to provide further explanation of the invention as claimed. Other objects, advantages and novel features will be readily apparent to those skilled in the art from the following detailed description of the invention.

### DETAILED DESCRIPTION

### Definitions

As used herein, unless otherwise stated, the singular forms “a,” “an,” and “the” include plural reference. Thus, for example, a reference to “a cell” includes a plurality of cells, and a reference to “a molecule” is a reference to one or more molecules.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The term “alkyl” refers to a straight or branched chain, saturated hydrocarbon having the indicated number of carbon atoms. For example, (C₁-C₁₀)alkyl is meant to include but is not limited to methyl, ethyl, propyl, isopropyl, butyl, sec-butyl, tert-butyl, pentyl, isopentyl, neopentyl, hexyl, isohexyl, and neohexyl, etc. An alkyl group can be unsubstituted or optionally substituted with one or more substituents as described herein below.

The term “alkenyl” refers to a straight or branched chain unsaturated hydrocarbon having the indicated number of carbon atoms and at least one double bond. Examples of a (C₂-C₁₀)alkenyl group include, but are not limited to, ethylene, propylene, 1-butylene, 2-butene, isobutene, sec-butene, 1-pentene, 2-pentene, isopentene, 1-hexene, 2-hexene, 3-hexene, isohexene, 1-heptene, 2-heptene, 3-heptene, isoheptene, 1-octene, 2-octene, 3-octene, 4-octene, and isooctene. An alkenyl group can be unsubstituted or optionally substituted with one or more substituents as described herein below.

The term “alkynyl” refers to a straight or branched chain unsaturated hydrocarbon having the indicated number of carbon atoms and at least one triple bond. Examples of a (C₂-C₁₀)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below.

The term “alkoxy” refers to an —O-alkyl group having the indicated number of carbon atoms. For example, a (C₁-C₆)alkoxy group includes —O-methyl, —O-ethyl, —O-propyl, —O— isopropyl, —O-butyl, —O-sec-butyl, —O-tert-butyl, —O-pentyl, —O-isopentyl, —O-neopentyl, —O— hexyl, —O-isohexyl, and —O-neohexyl.

The term “aryl” refers to a 3- to 14-member monocyclic, bicyclic, tricyclic, or polycyclic aromatic hydrocarbon ring system. Examples of an aryl group include naphthyl, pyrenyl, and anthracyl. An aryl group can be unsubstituted or optionally substituted with one or more substituents as described herein below.

The terms “alkylene,” “cycloalkylene,” “alkenylene.” “alkynylene,” “arylene,” and “heteroarylene,” alone or as part of another substituent, means a divalent radical derived from an alkyl, cycloalkyl, alkenyl, alkynyl, aryl, or heteroaryl group, respectively, as exemplified by —CH₂CH₂CH₂CH₂—. For alkylene, alkenylene, or aryl linking groups, no orientation of the linking group is implied.

The term “halogen” and “halo” refers to —F, —Cl, —Br or —I.

The term “heteroatom” is meant to include oxygen (O), nitrogen (N), and sulfur (S).

A “hydroxyl” or “hydroxy” refers to an —OH group.

The term “hydroxyalkyl,” refers to an alkyl group having the indicated number of carbon atoms wherein one or more of the alkyl group's hydrogen atoms is replaced with an —OH group. Examples of hydroxyalkyl groups include, but are not limited to, —CH₂OH, —CH₂CH₂OH, —CH₂CH₂CH₂OH, —CH₂CH₂CH₂CH₂OH, —CH₂CH₂CH₂CH₂CH₂OH, —CH₂CH₂CH₂CH₂CH₂CH₂OH, and branched versions thereof.

The term “cycloalkyl” or “carbocycle” refer to monocyclic, bicyclic, tricyclic, or polycyclic, 3- to 14-membered ring systems, which are either saturated, unsaturated or aromatic. The heterocycle may be attached via any heteroatom or carbon atom. Cycloalkyl include aryls and hetroaryls as defined above. Representative examples of cycloalky include, but are not limited to, cycloethyl, cyclopropyl, cycloisopropyl, cyclobutyl, cyclopentyl, cyclohexyl, cyclopropene, cyclobutene, cyclopentene, cyclohexene, phenyl, naphthyl, anthracyl, benzofuranyl, and benzothiophenyl. A cycloalkyl group can be unsubstituted or optionally substituted with one or more substituents as described herein below.

The term “nitrile or cyano” can be used interchangeably and refer to a —CN group which is bound to a carbon atom of a heteroaryl ring, aryl ring and a heterocycloalkyl ring.

The term “amine or amino” refers to an —NR_{c}R_{d} group wherein R_{c} and R_{d} each independently refer to a hydrogen, (C₁-C₈)alkyl, aryl, heteroaryl, heterocycloalkyl, (C₁-C₈)haloalkyl, and (C₁-C₆)hydroxyalkyl group.

The term “TMS” refers to a trimethyl silyl group.

The term “TBDMS” refers to a t-butyldimethylsilyl group.

The terms “benzyl” or “Bz” refer to a benzyl group, that is, a C₆H₅—CH₂— group.

The term “THP” refers to the tetrahydropyran group.

The term “alkylaryl” refers to C₁-C₈ alkyl group in which at least one hydrogen atom of the C₁-C₈ alkyl chain is replaced by an aryl atom, which may be optionally substituted with one or more substituents as described herein below. Examples of alkylaryl groups include, but are not limited to, methylphenyl, ethylnaphthyl, propylphenyl, and butylphenyl groups.

“Arylalkylene” refers to a divalent alkylene wherein one or more hydrogen atoms in the C₁-C₁₀ alkylene group is replaced by a (C₃-C₁₄)aryl group. Examples of (C₃-C₁₄)aryl-(C₁-C₁₀)alkylene groups include without limitation 1-phenylbutylene, phenyl-2-butylene, 1-phenyl-2-methylpropylene, phenylmethylene, phenylpropylene, and naphthylethylene.

“Arylalkenylene” refers to a divalent alkenylene wherein one or more hydrogen atoms in the C₂-C₁₀ alkenylene group is replaced by a (C₃-C₁₄)aryl group.

The term “arylalkynylene” refers to a divalent alkynylene wherein one or more hydrogen atoms in the C₂-C₁₀ alkynylene group is replaced by a (C₃-C₁₄)aryl group.

The terms “carboxyl” and “carboxylate” include such moieties as may be represented by the general formulas:

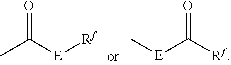

E in the formula is a bond or O and R^{f} individually is H, alkyl, alkenyl, aryl, or a pharmaceutically acceptable salt. Where E is O, and R^{f} is as defined above, the moiety is referred to herein as a carboxyl group, and particularly when R^{f} is a hydrogen, the formula represents a “carboxylic acid”. In general, where the expressly shown oxygen is replaced by sulfur, the formula represents a “thiocarbonyl” group.

Unless otherwise indicated. “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

If there is a discrepancy between a depicted structure and a name given that structure, then the depicted structure controls. Additionally, if the stereochemistry of a structure or a portion of a structure is not indicated with, for example, bold or dashed lines, the structure or portion of the structure is to be interpreted as encompassing all stereoisomers of it.

The present invention focuses on a prodrug of a cannabinoid or a cannabinoid analog as well as biosynthetic methodologies for the manufacture of a prodrug of a cannabinoids or a cannabinoid analog. More specifically, the invention relates to enzyme-catalyzed synthesis of a prodrug form of a cannabinoid or cannabinoid analog in a cell-free environment.

The term “prodrug” refers to a precursor of a biologically active pharmaceutical agent (drug). Prodrugs must undergo a chemical or a metabolic conversion to become a biologically active pharmaceutical agent. A prodrug can be converted ex vivo to the biologically active pharmaceutical agent by chemical transformative processes. In vivo, a prodrug is converted to the biologically active pharmaceutical agent by the action of a metabolic process, an enzymatic process or a degradative process that removes the prodrug moiety to form the biologically active pharmaceutical agent.

Accordingly, in one of its embodiments the present invention provides a method for producing a cannabinoid prodrug according to Formula II or Formula III:

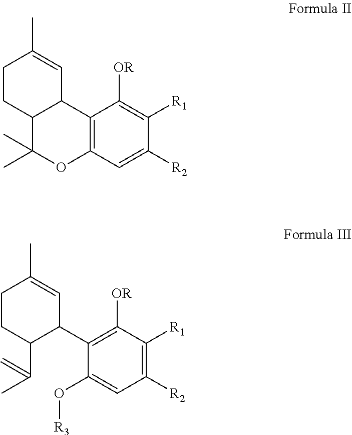

by contacting a compound according to Formula I

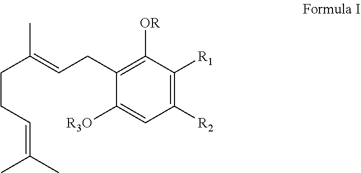

with the cannabinoid synthase to produce a compound according to Formula II or Formula III.

For Formula I, II, and III compounds substituents R and R³ are each independently selected from the group consisting of —H, acetyl, propionyl, 3-hydroxy-2-methylpropionyl, TMS, TBDMS, benzyl, tetrahydropyranyl, —C(O)[CH₂]ₓ—C(O)OH, —C(O)[CH₂]ₓ—OR⁴, —C(O)[CHR₄]ₓ—C(O)OH, —C(O)[CHR⁴]ₓ—OR⁵, —C(O)[CR⁴R⁵]ₓ—OR⁶, —C(O)O[CH₂]ₓ—OR⁴, —C(O)—CH₂—[OCH₂CH₂]ₓ—OR⁴, —C(O)—C(O)—[OCH₂H₂C₂]ₓ—OR⁴, —C(O)[CH₂]ₓ—NR⁴R⁵, —C(O)O[CH₂]ₓ—NR⁴R⁵, —C(O)—NH—[CH₂]ₓ—NR⁴R⁵, —C(O)[CH₂]ₓ—N⁺(R⁴)(R⁵))(R⁶)X⁻, —C(O)O[CH₂]ₓ—N⁺(R⁴)(R⁵))(R⁶)X⁻, —C(O)—NH—[CH₂]ₓ—N⁺(R⁴)(R⁵))(R⁶)X⁻, a L-amino acid residue, a D-amino acid residue, a ß-amino acid residue, a γ-amino acid residue, —P(O)[OY](OZ), and —P(O)[NR⁴NR⁵][OY](OZ).

For certain Formula I, II, and III compounds substituent R¹ is —H, —COOH, —COOMe, —COOEt, or —COO(t-Bu) and R² is selected from the group consisting of (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene. Thus, the invention provides in one embodiment Formula I, II, and III compounds where R¹ is —COOH and R² is a (C₁-C₁₀)alkyl, for instance, methyl, ethyl, propyl, butyl, or pentyl.

In one embodiment, the invention provides a Formula II compound where substituent R is —C(O)[CH₂]ₓ—OR⁴, —C(O)[CHR⁴]ₓ—OR⁵, —C(O)[CR⁴R⁵]ₓ—OR⁶, or —C(O)O[CH₂]ₓ—OR⁴, R¹ is —COOH, and R² is a (C₁-C₁₀)alkyl, for instance, propyl, or pentyl.

For such Formula II compounds, substituents R⁴, R⁵, and R⁶ are each independently selected from the group consisting of —H, —OH, formyl, acetyl, pivaloyl, —NH₂, —NH(CH₃), —NH(CH₂CH₃), —N(CH₃)₂, —NH[C(O)H], —NH[C(O)CH₃], and (C₁-C₅)alkyl.

According to this embodiment, when R is —C(O)[CH₂]ₓ—OR⁴, or —C(O)O[CH₂]ₓ—OR⁴, substituent R⁴ is —H, methyl, or ethyl and subscript “x” is 1, 2, 3, 4, 5, or 6. In one embodiment, R⁴ is —H and subscript “x” is 1, or 2. According to another embodiment, R⁴ is —CH₃ and subscript “x” is 1, or 2.

For some of the inventive Formula II compounds, R is —C(O)[CHR⁴]ₓ—OR⁵, R¹ is —COOH or —COOEt, R² is propyl or pentyl, and subscript “x” is 1, or 2. In one embodiment, R⁴ is —OH and R⁵ is —H, methyl, or ethyl. Thus, the invention provides a method for producing a cannabinoid prodrug according to Formula II where substituent R is —C(O)—CH(OH)—CH₂—OH, R¹ is —COOH and R² is propyl or pentyl.

For some prodrugs according to Formula II substituent R is —C(O)[CH₂]ₓ—NR⁴R⁵, —C(O)—NH—[CH₂]ₓ—NR⁴R⁵, —C(O)O[CH₂]ₓ—N⁺(R⁴)(R⁵)(R⁶)X⁻, R¹ is —COOH or —COOEt, and R² is a (C₁-C₁₀)alkyl, for instance, propyl, or pentyl.

In one embodiment, R is —C(O)O[CH₂]ₓ—N⁺(R⁴)(R⁵))(R⁶)X⁻, R¹ is —COOH or —COOEt, and R² is propyl, or pentyl. For such Formula II prodrugs, R⁴, R⁵, and R⁶ are each independently —H, methyl, ethyl, or a combination thereof, and X⁻ is a counter-ion, such as chloride, bromide, phosphate, acetate, citrate, sulfate, succinate, hemisuccinate, oxalate, or malonate. For such prodrugs, subscript “x” is 1, 2, 3, or 4.

According to another aspect, for compounds in accordance with Formula II, R is —C(O)[CH₂]ₓ—NR⁴R⁵, R¹ is —COOH or —COOEt, and R² is propyl, or pentyl. Substituents R⁴ and R⁵ for such compounds are each independently —H, methyl, ethyl, acetyl, or formyl and subscript “x” is 1, 2, 3, or 4.

In yet another embodiment, R is —C(O)—NH—[CH₂]ₓ—NR⁴R⁵ and each of R⁴ and R⁵ is —H, methyl, ethyl, acetyl, or formyl. Illustrative of such prodrugs without limitation are Formula II compounds where R is —C(O)—NH—[CH₂]—NH₂, —C(O)—NH—[CH₂]—N(CH₃)₂, —C(O)—NH—[CH₂]—NH(CH₃), —C(O)—NH—[CH₂]—NH(formyl), and —C(O)—NH—[CH₂]—NCH₃(formyl).

In one embodiment, the prodrug of Formula II is one in which R is —C(O)—CH₂—[OCH₂CH₂]ₓ—OR⁴, —C(O)—C(O)—[OCH₂CH₂]ₓ—OR⁴, R¹ is —COOH, and R² is propyl, or pentyl. Illustrative of such R groups without limitation are —C(O)—CH₂—[OCH₂CH₂]₂—OH, —C(O)—CH₂—[OCH₂CH₂]₂—OCH₃, —C(O)—CH₂—[OCH₂CH₂]₃—OH, and —C(O)—CH₂—[OCH₂CH₂]₂—OCH₃.

The cannabinoid prodrugs according to Formula II described above can optionally be decarboxylated prior to their use as a pharmaceutical agent. Decarboxylation is achieved by any physical or chemical means that maintains the pharmacological integrity of the inventive prodrug, for example, by contacting the Formula II prodrug that has a carboxylic acid group at R¹ with a source of heat or UV-light. Alternatively, de-carboxylation is achieved by contacting a solution of such a compound with a weak base, for example with sodium bicarbonate.

Illustrative of Formula II prodrugs that are de-carboxylated using a protocol described above are those where R¹ is —COOH, R² is propyl or pentyl, and substituent R is one of —C(O)[CH₂]—OH, —C(O)[CH₂]₂—OH, —C(O)[CH₂]—OCH₃, —C(O)[CH₂]₂—OCH₃, —C(O)—CH(OH)—CH₂—OH, —C(O)O[CH₂]—N⁺(CH₂CH₃)₂(CH₃)X⁻, —C(O)O[CH₂]—N⁺(CH₂CH₃)₃X⁻, —C(O)O[CH₂]—N⁺(CH₃)₃X⁻, —C(O)O[CH₂]₂—N⁺(CH₂CH₃)₂(CH₃)X⁻, —C(O)O[CH₂]₂—N⁺(CH₂CH₃)₃X⁻, —C(O)O[CH₂]₂—N⁺(CH₃)₃X⁻, —C(O)NH[CH₂]—N⁺(CH₂CH₃)₂(CH₃)X⁻, —C(O)NH[CH₂]—N⁺(CH₂CH₃)₃X⁻, —C(O)NH[CH₂]—N⁺(CH₃)₃X⁻, —C(O)NH[CH₂]₂—N⁺(CH₂CH₃)₂(CH₃)X⁻, —C(O)NH[CH₂]₂—N⁺(CH₂CH₃)₃X⁻, or —C(O)NH[CH₂]₂—N⁺(CH₃)₃X⁻.

According to yet another embodiment, the de-carboxylated Formula II prodrugs are compounds where R¹ is —H, R² is propyl or pentyl and substituent R is a polyethylene glycol group, for example —C(O)—CH₂—[OCH₂CH₂]₂—OH, —C(O)—CH₂—[OCH₂CH₂]₂—OCH₃, —C(O)—CH₂—[OCH₂CH₂]₃—OH, or —C(O)—CH₂—[OCH₂CH₂]₃—OCH₃.

Table 1 structurally illustrates exemplary Formula II prodrugs produced using the inventive method, where X⁻ is a counter ion as described above.

TABLE 1

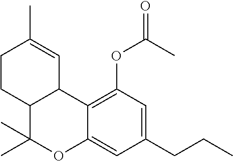 [SMILES: CCCc1cc(OC(C)=O)c2c(c1)OC(C)(C)C1CCC(C)=CC21]

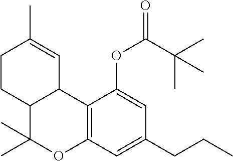 [SMILES: CCCc1cc(OC(=O)C(C)(C)C)c2c(c1)OC(C)(C)C1CCC(C)=CC21]

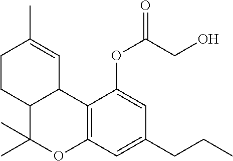 [SMILES: CCCc1cc(OC(=O)CO)c2c(c1)OC(C)(C)C1CCC(C)=CC21]

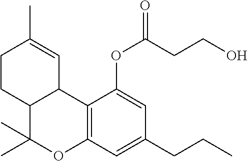 [SMILES: CCCc1cc(OC(=O)CCO)c2c(c1)OC(C)(C)C1CCC(C)=CC21]

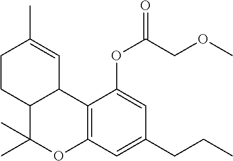 [SMILES: CCCc1cc(OC(=O)COC)c2c(c1)OC(C)(C)C1CCC(C)=CC21]

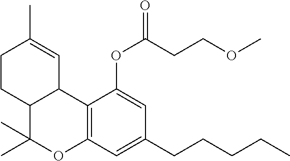 [SMILES: CCCCCc1cc(OC(=O)CCOC)c2c(c1)OC(C)(C)C1CCC(C)=CC21]

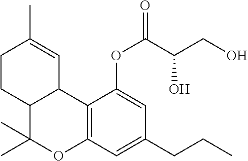 [SMILES: CCCc1cc(OC(=O)[C@@H](O)CO)c2c(c1)OC(C)(C)C1CCC(C)=CC21]

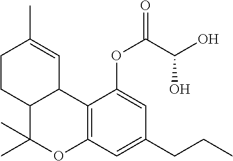

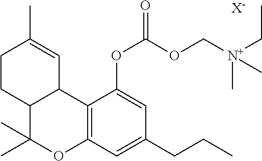

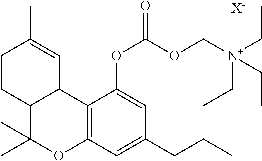

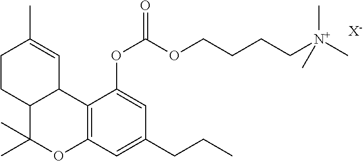

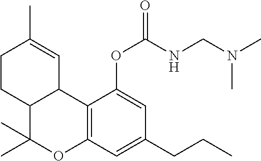 [SMILES: CCCc1cc(OC(=O)NCN(C)C)c2c(c1)OC(C)(C)C1CCC(C)=CC21]

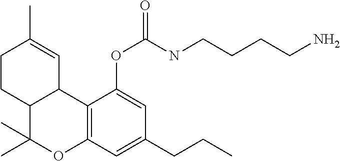 [SMILES: CCCc1cc(OC(=O)NCCCCN)c2c(c1)OC(C)(C)C1CCC(C)=CC21]

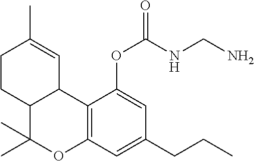 [SMILES: CCCc1cc(OC(=O)NCN)c2c(c1)OC(C)(C)C1CCC(C)=CC21]

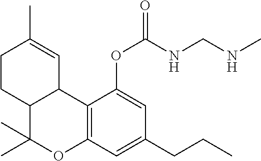 [SMILES: CCCc1cc(OC(=O)NCNC)c2c(c1)OC(C)(C)C1CCC(C)=CC21]

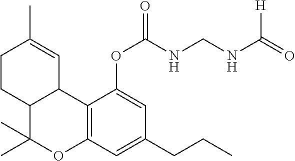 [SMILES: CCCc1cc(OC(=O)NCNC=O)c2c(c1)OC(C)(C)C1CCC(C)=CC21]

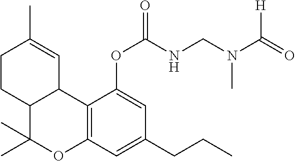 [SMILES: CCCc1cc(OC(=O)NCN(C)C=O)c2c(c1)OC(C)(C)C1CCC(C)=CC21]

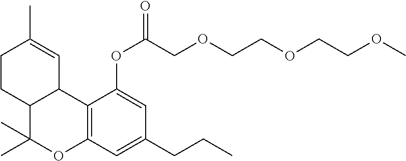 [SMILES: CCCc1cc(OC(=O)COCCOCCOC)c2c(c1)OC(C)(C)C1CCC(C)=CC21]

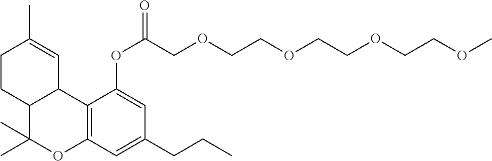 [SMILES: CCCc1cc(OC(=O)COCCOCCOCCOC)c2c(c1)OC(C)(C)C1CCC(C)=CC21]

The inventive method also permits the synthesis of a cannabinoid prodrug according to Formula III. These prodrugs can be de-carboxylated, if necessary, prior to their use as pharmaceutical agents using one of the protocols described above.

Accordingly, in one embodiment, the prodrug according to Formula III is a compound where substituent R is —C(O)[CH₂]—OH, —C(O)[CH₂]₂—OH, —C(O)[CH₂]—OCH₃, —C(O)[CH₂]₂—OCH₃, or —C(O)—CH(OH)—CH₂—OH, substituent R¹ is —COOH, —COOMe, or —COOEt, R² is propyl or pentyl, and R³ is —H, TMS, TBDMS, tetrahydropyran, or benzyl.

According to another embodiment, the prodrug according to Formula III is a compound where substituents R and R³ are each independently —C(O)[CH₂]—OH, —C(O)[CH₂]₂—OH, —C(O)[CH₂]—OCH₃, —C(O)[CH₂]₂—OCH₃, and —C(O)—CH(OH)—CH₂—OH; substituent R¹ is —H or —COOH, and R² is propyl or pentyl.

In one embodiment, the prodrug according to Formula III is a compound where substituent R is —C(O)O[CH₂]—N⁺(CH₂CH₃)₂(CH₃)X⁻, —C(O)O[CH₂]—N⁺(CH₂CH₃)₃X⁻, —C(O)O[CH₂]—N⁺(CH₃)₃X⁻, —C(O)O[CH₂]₂—N⁺(CH₂CH₃)₂(CH₃)X⁻, —C(O)O[CH₂]₂—N⁺(CH₂CH₃)₃X⁻, or —C(O)O[CH₂]₂—N⁺(CH₃)₃X⁻, substituent R¹ is —COOH or —COOEt, and R² is propyl or pentyl. Such a Formula III prodrug is decarboxylated if necessary prior to its use as a pharmaceutical agent.

According to one aspect of this embodiment, the prodrug according to Formula III is a compound where both R and R³ are —C(O)O[CH₂]—N⁺(CH₂CH₃)₂(CH₃)X⁻, —C(O)O[CH₂]N⁺(CH₂CH₃)₃X⁻, —C(O)O[CH₂]N⁺(CH₃)₃X⁻, —C(O)O[CH₂]₂—N⁺(CH₂CH₃)₂(CH₃)X⁻, —C(O)O[CH₂]₂—N⁺(CH₂CH₃)₃X⁻, or —C(O)O[CH₂]₂—N⁺(CH₃)₃X⁻ and substituent R¹ is —H or —COOH.

For certain Formula III prodrugs, R is —C(O)NH[CH₂]—N⁺(CH₂CH₃)₂(CH₃)X⁻, —C(O)NH[CH₂]—N—(CH₂CH₃)₃X⁻, —C(O)NH[CH₂]—N(CH₃)₃X⁻, —C(O)NH[CH₂]₂—N⁺(CH₂CH₃)₂(CH₃)X⁻, —C(O)NH[CH₂]₂—N⁺(CH₂CH₃)₃X⁻, or —C(O)NH[CH₂]₂—N⁺(CH₃)₃X⁻. Alternatively, both R and R³ are —C(O)NH[CH₂]—N⁺(CH₂CH₃)₂(CH₃)X⁻, —C(O)NH[CH₂]—N⁺(CH₂CH₃)₃X⁻, —C(O)NH[CH₂]—N⁺(CH₃)₃X⁻, —C(O)NH[CH₂]₂—N⁺(CH₂CH₃)₂(CH₃)X⁻, —C(O)NH[CH₂]₂—N⁺(CH₂CH₃)₃X⁻, or —C(O)NH[CH₂]₂—N⁺(CH₃)₃X⁻, R¹ is —H or —COOH and R² is propyl or pentyl.

For such prodrugs, X⁻ is a counter-ion, such as chloride, bromide, phosphate, acetate, citrate, sulfate, succinate, hemisuccinate, oxalate, or malonate.

When R¹ is —COOH, the Formula III prodrug can be decarboxylated prior to its use as a pharmaceutical agent. De-carboxylation proceeds by contacting the prodrug with heat or exposing a solution of the prodrug to UV-light or by contact with a solution of a base such as sodium bicarbonate.

For any Formula III compound, such as the ones described above, when R³ is TMS, benzyl, or TBDMS in Formula III, these protecting groups are removed using protocols well known in the chemical art prior to their utilization as pharmaceutical agents.

Exemplary Formula III prodrugs produced using the inventive method are those shown in Table 2.

TABLE 2

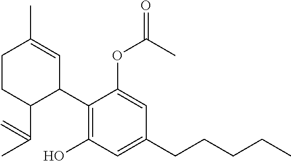 [SMILES: C=C(C)C1CCC(C)=CC1c1c(O)cc(CCCCC)cc1OC(C)=O]

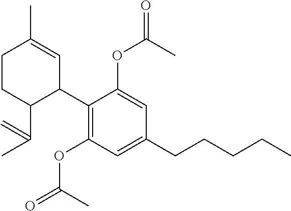 [SMILES: C=C(C)C1CCC(C)=CC1c1c(OC(C)=O)cc(CCCCC)cc1OC(C)=O]

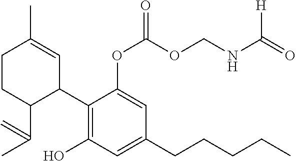 [SMILES: C=C(C)C1CCC(C)=CC1c1c(O)cc(CCCCC)cc1OC(=O)OCNC=O]

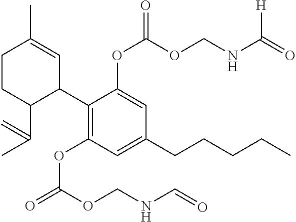 [SMILES: C=C(C)C1CCC(C)=CC1c1c(OC(=O)OCNC=O)cc(CCCCC)cc1OC(=O)OCNC=O]

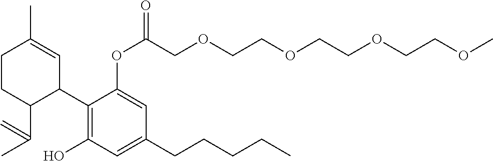 [SMILES: C=C(C)C1CCC(C)=CC1c1c(O)cc(CCCCC)cc1OC(=O)COCCOCCOCCOC]

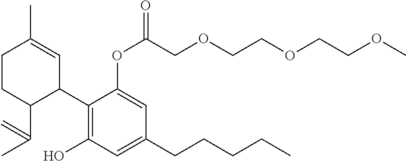 [SMILES: C=C(C)C1CCC(C)=CC1c1c(O)cc(CCCCC)cc1OC(=O)COCCOCCOC]

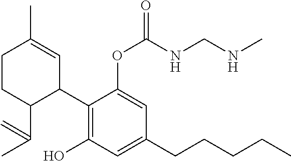 [SMILES: C=C(C)C1CCC(C)=CC1c1c(O)cc(CCCCC)cc1OC(=O)NCNC]

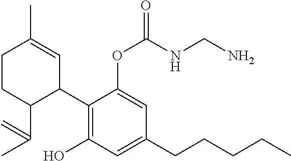 [SMILES: C=C(C)C1CCC(C)=CC1c1c(O)cc(CCCCC)cc1OC(=O)NCN]

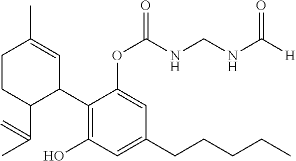 [SMILES: C=C(C)C1CCC(C)=CC1c1c(O)cc(CCCCC)cc1OC(=O)NCNC=O]

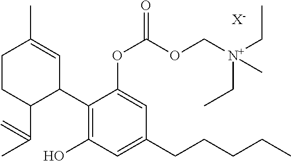

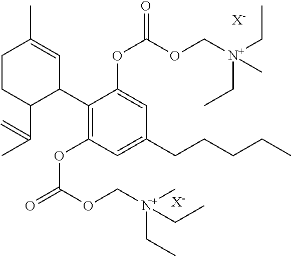

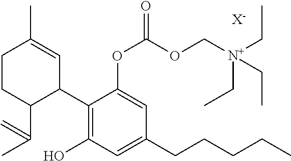

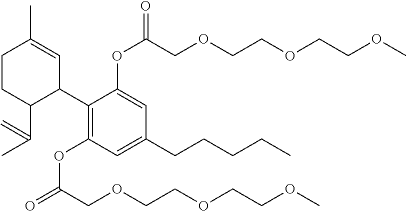 [SMILES: C=C(C)C1CCC(C)=CC1c1c(OC(=O)COCCOCCOC)cc(CCCCC)cc1OC(=O)COCCOCCOC]

Cannabinoid acid synthase enzymes used to synthesize a cannabinoid prodrug according to the inventive method include without limitation tetrahydrocannabinolic acid synthase (THCA synthase), tetrahydrocannabivarin acid synthase (THCVA synthase), cannabidiolic acid synthase (CBDA synthase), or cannabichromene acid synthase (CBCA synthase). These enzymes may be obtained from natural sources or may be obtained by using any suitable recombinant method, including the use of the PichiaPink™ Yeast Expression system described in U.S. Provisional Application No. 62/041,521, filed Aug. 25, 2014 and U.S. patent application Ser. No. 14/835,444, filed Aug. 25, 2015 which published as U.S. Publication No.: 2016-0053220 on Feb. 26, 2016, the contents of which applications are incorporated by reference in their entireties.

In one embodiment of the invention, the solvent used to produce a prodrug using the inventive method is an aqueous buffer, a non-aqueous solvent, or a mixture comprising an aqueous buffer and a non-aqueous solvent. Buffers typically used in the method of the invention are citrate buffer, phosphate buffer, HEPES, Tris buffer, MOPS, or glycine buffer. Illustrative non-aqueous solvents include without limitation dimethyl sulfoxide (DMSO), dimethyl formamide (DMF), or iso-propyl alcohol, ß-cyclodextrin, and combinations thereof.

In one embodiment the solvent is a mixture of a aqueous buffer and a non-aqueous solvent. For such mixtures, the concentration of the non-aqueous solvent can vary between 10% and 50% (v/v), preferably the concentration of the non-aqueous solvent in the reaction mixture is 10%, 12%, 15%, 20%, 25%, 30%, 35%, 40%, 45%, or 50%. In one embodiment the concentration of the non-aqueous solvent in the reaction mixture is 30%. In another embodiment, the concentration of the non-aqueous solvent in the reaction mixture is 20%, or may vary between 10% and 20%, between 10% and 30%, or between 10% and 40%.

The inventors of the present application have unexpectedly discovered that the concentration of the non-aqueous solvent in the reaction mixture affects the rate of the enzyme-catalyzed reaction as well as the ratio of the cannabinoid prodrug obtained as products. For example, it was observed that the presence of cyclodextrins, cyclic oligosaccharides that are amphiphilic and function as surfactants, accelerates the rate of the enzyme catalyzed cyclization reaction of a Formula I compound (substrate) to a Formula II or Formula III compound (product). It was surprising to note that the concentration of cyclodextrin in the reaction mixture also affects product ratio, that is, the ratio of the amount of a Formula II compound to the amount of a Formula III compound produced using the inventive method.

Another surprising and unexpected observation was that pH of the reaction mixture affects the ratio of the cannabinoid prodrugs produced using the inventive method. In one preferred embodiment, a Formula I compound according to the invention when contacted with THCA synthase produces a prodrug of a tetrahydrocannabinolic acid (THCA) or a prodrug of a cannabichromene acid (CBCA), in different ratios depending on the pH of the reaction mixture.

Thus in one its embodiments the invention provides a method for producing cannabinoid prodrugs at varying pH values of the reaction mixture. In one example, the bioenzymatic synthesis of a prodrug is performed at a pH in a range between 3.0 and 8.0, for example at a pH in a range between 3.0 and 7.0, between 3.0 and 6.0, between 3.0 and 5.0, or between 3.0 and 4.0.

In one embodiment, the reaction is performed at a pH in a range between 3.8 and 7.2. According to another embodiment, the reaction is performed at a pH in a range between 3.5 and 8.0, between 3.5 and 7.5, between 3.5 and 7.0, between 3.5 and 6.5, between 3.5 and 6.0, between 3.5 and 5.5, between 3.5 and 5.0, or between 3.5 and 4.5.

The invention also provides cannabinoid prodrugs according to Formula IV or Formula V.

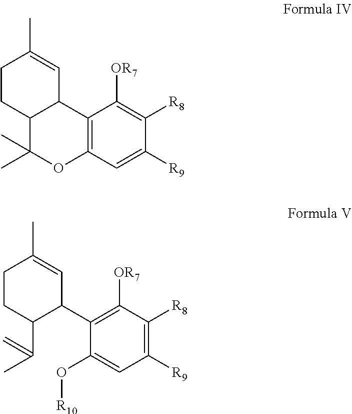

For Formula IV or Formula V prodrugs, R⁷ or R¹⁰ are each independently selected from the group consisting of —H, acetyl, propionyl, 3-hydroxy-2-methylpropionyl, —C(O)[CH₂]ₓ—C(O)OH, —C(O)[CH₂]ₓ—OR¹¹, —C(O)[CHR¹¹]ₓ—C(O)OH, —C(O)[CHR¹¹]ₓ—OR¹², —C(O)[CR¹¹R¹²]ₓ—OR¹¹, —C(O)O[CH₂]ₓ—OR¹¹, —C(O)—CH₂—[OCH₂CH₂]ₓ—OR¹¹, —C(O)—C(O)—[OCH₂CH₂]ₓ—OR¹¹, —C(O)[CH₂]ₓ—NR¹¹R¹², —C(O)O[CH₂]ₓ—NR¹¹R¹², —C(O)—NH—[CH₂]ₓ—NR¹¹R¹², —C(O)[CH₂]ₓ—N⁺(R¹¹)(R¹²))(R¹³)X⁻, —C(O)O[CH₂]ₓ—N⁺(R¹¹)(R¹²))(R¹³)X⁻, —C(O)—NH—[CH₂]ₓ—N⁺(R¹¹)(R¹²))R¹³)X⁻, a L-amino acid residue, a D-amino acid residue, a ß-amino acid residue, a γ-amino acid residue, —P(O)[OY](OZ), and —P(O)[NR¹¹R¹²][OY]. Subscripts “x” and “n” are independently selected from the group consisting of 0, 1, 2, 3, 4, 5, and 6. In various embodiments, substituents R¹¹, R¹² and R¹³ are each independently —H or a (C₁-C₅)alkyl, for example, methyl, ethyl propyl, butyl or t-butyl. For certain other compounds, substituents R¹¹ and R¹² are selected from —NH₂, —NH(CH₃), —NH(CH₂CH₃), or N(CH₃)₂.

Exemplary ß-amino acid residues according to the present invention include without limitation ß-phenylalanine, ß-alanine, 3-aminobutanoic acid, 3-amino-3(3-bromophenyl)propionic acid, 2-amino-3-cyclopentene-1-carboxylic acid, 3-aminoisobutyric acid, 3-amino-2-phenylpropionic acid, 4,4-biphenylbutyric acid, 3-aminocyclohexanecarboxylic acid, 3-aminocyclopentanecarboxylic acid, and 2-aminoethylphenylacetic acid.

Illustrative γ-amino acids include without limitation γ-aminobutyric acid, statine, 4-amino-3-hydroxybutanoic acid, and 4-amino-3-phenylbutanoic acid (baclofen).

For Formula IV or Formula V prodrugs, substituent R⁸ is —H, —COOH, or —COOR^{a}, or —(CH₂)ₙCOOH and substituent R⁹ in Formula IV and V is selected from the group consisting of (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene.

When R⁸ is —COOR^{a}, substituent R^{a} is selected from (C₁-C₁₀)alkyl, such as methyl, ethyl, propyl, or t-butyl. In one embodiment R^{a} is ethyl or t-butyl.

For prodrugs in accordance with the invention, substituents R¹¹, R¹² and R¹³ are each independently selected from the group consisting of —H, —OH, formyl, acetyl, pivaloyl, —NH₂, —NH(CH₃), —NH(CH₂CH₃), N(CH₃)₂, —NH[C(O)H], —NH[C(O)CH₃], and (C₁-C₅)alkyl, variable “X” is a counter ion derived from a pharmaceutically acceptable acid while variables “Y” and “Z” are each independently selected from the group consisting of —H, (C₁-C₅)alkyl, alkali metal cations, alkaline earth metal cations, ammonium cation, methyl ammonium cation, and cations obtained from pharmaceutically acceptable bases. Subscripts “x” and “n” for Formula IV and V prodrugs are any integer, such as 0, 1, 2, 3, 4, 5, or 6.

Exemplary pharmaceutically acceptable acids include without limitation formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, beta-hydroxybutyric, galactaric and galacturonic acids. The list of pharmaceutically acceptable salts mentioned above is not meant to be exhaustive but merely illustrative, because a person of ordinary skill in the art would appreciate that other pharmaceutically acceptable salts of a prodrug of a cannabinoid and can be prepared using methods known in the formulary arts.

For example, acid addition salts are readily prepared from a free base by reacting the free base with a suitable acid. Suitable acids for preparing acid addition salts include both (i) organic acids, for example, formic acid, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, and (ii) inorganic acids, for example, hydrochloric acid, hydrobromic acid, sulfuric acid, nitric acid, phosphoric acid, and the like.

In one embodiment, for Formula IV and V prodrugs, R¹¹, R¹² and R¹³ are each independently selected from —H or (C₁-C₅)alkyl. When any of R¹¹, R¹² or R¹³ are (C₁-C₅)alkyl, the alkyl group is selected from methyl, ethyl, propyl, butyl, pentyl, or combinations thereof. In aspect of this embodiment, R¹¹, R¹² and R¹³ are each independently selected from —H, methyl, or ethyl.

In one embodiment, R⁷ is acetyl, propionyl, 3-hydroxy-2-methylpropionic acid, R⁸ is —COOH, substituent R⁹ is a (C₁-C₁₀)alkyl, and R¹⁰ is —H.

According to another embodiment, each of R⁷ and R¹⁰ are each independently acetyl, propionyl, 3-hydroxy-2-methylpropionic acid, R⁸ is —COOH, and substituent R⁹ is a (C₁-C₁₀)alkyl, for example, methyl, propyl or pentyl.

For some Formula IV and V compounds, R⁷ is selected from the group consisting of —C(O)[CH₂]ₓ—C(O)OH, —C(O)[CH₂]ₓ—OR¹¹, —C(O)[CH₂]ₓ—NR¹¹R¹², —C(O)—CH₂—[OCH₂CH₂]ₓ—OR¹¹, and —C(O)[CH₂]ₓ—N⁺(R¹¹)(R¹²)(R¹³)X⁻.

According to one embodiment, R⁷ and R¹¹ are each independently —C(O)[CH₂]ₓ—C(O)OH, —C(O)[CH₂]ₓ—OR¹¹, —C(O)[CH₂]ₓ—NR¹¹R¹², —C(O)—CH₂—[OCH₂CH₂]ₓ—OR¹¹, or —C(O)[CH₂]ₓ—N⁺(R¹¹)(R¹²)¹³)X⁻.

If R⁸ is —COOH, the Formula IV or Formula V prodrug can be de-carboxylated prior to its use as a pharmaceutical agent. De-carboxylation is achieved by contacting the Formula IV or Formula V prodrug in acid form with heat, or contacting a solution of the prodrug acid with heat or UV-light.

In one embodiment, R⁸ is —H, and R⁹ is propyl or pentyl for prodrugs according to Formula IV. Substituent R⁷ according to this embodiment is a group selected from acetyl, pivaloyl, 2-hydroxyacetyl, —C(O)[CH₂]₂—OH, —C(O)[CH₂]—OCH₃, —C(O)[CH₂]₂—OCH₃, —C(O)[CH(OH)—CH₂]—OH, and —C(O)[CH(OH)]—OH.

According to another embodiment, both R⁷ and R¹⁰ are chemical moieties selected from the group consisting of acetyl, pivaloyl, 2-hydroxyacetyl, —C(O)[CH₂]₂—OH, —C(O)[CH₂]—OCH₃, —C(O)[CH₂]₂—OCH₃, —C(O)[CH(OH)—CH₂]—OH, and —C(O)[CH(OH)]—OH.

In one embodiment, R⁷ is acetyl and R¹⁰ is 2-hydroxyacetyl. In another embodiment, R⁷ is acetyl and R¹⁰ is —C(O)[CH₂]₂—OH, or —C(O)[CH₂]—OCH₃.

In yet another embodiment, R⁷ is —C(O)[CH(OH)—CH₂]—OH and R¹⁰ is acetyl.

In yet another embodiment, R⁷ is —H and R¹⁰ is selected from the group consisting of acetyl, pivaloyl, 2-hydroxyacetyl, —C(O)[CH₂]₂—OH, —C(O)[CH₂]—OCH₃, —C(O)[CH₂]₂—OCH₃, —C(O)[CH(OH)—CH₂]—OH, and —C(O)[CH(OH)]—OH.

In one embodiment, R⁷ is —H and R¹⁰ is acetyl. In another embodiment, R⁷ is —H and R¹⁰ is —C(O)[CH₂]₂—OH, or —C(O)[CH₂]—OCH₃.

In one embodiment, R⁷ is —H and R¹⁰ is —C(O)[CH₂]₂—OCH₃. According to another embodiment, R⁷ is —H and R¹⁰ is —C(O)[CH(OH)—CH₂]—OH, or —C(O)[CH(OH)]—OH.

In one embodiment, substituent R⁷ is a group selected from —C(O)O[CH₂]—N⁺(CH₃)₃X⁻, —C(O)O[CH₂]—N⁺(Et)(CH₃)₂X⁻, —C(O)O[CH₂]—N⁺(CH₃(Et)₂X⁻, —C(O)O[CH₂]—N⁺(Et)₃X⁻, or —C(O)O[CH₂]₄—N⁺(CH₃)₃X⁻, R⁸ is —H, R⁹ is propyl and R¹⁰ is —H.

In one embodiment, R⁷ and R¹⁰ are both —C(O)O[CH₂]—N⁺(CH₃)₃X⁻, or —C(O)O[CH₂]—N⁺CH₃(Et)₂X⁻.

According to another embodiment, R⁷ and R¹⁰ are both —C(O)O[CH₂]—N⁺(Et)(CH₃)₂X⁻, or —C(O)O[CH₂]—N⁺(Et)₃X⁻. In yet another embodiment, R⁷ and R¹⁰ are both —C(O)O[CH₂]₄—N⁺(CH₃)₃X⁻.

According to another embodiment, substituent R⁷ is a group selected from —C(O)O[CH₂]—N⁺(CH₃)₃X⁻, —C(O)O[CH₂]—N⁺(Et)(CH₃)₂X⁻, —C(O)O[CH₂]—N⁺CH₃(Et)₂X⁻, —C(O)O[CH₂]—N⁺(Et)₃X⁻ or —C(O)O[CH₂]₄—N⁺(CH₃)₃X⁻, R⁹ is pentyl and R¹⁰ is —H.

According to another embodiment, R⁷ and R¹⁰ in Formula V are both —C(O)O[CH₂]—N⁺(CH₃)₃X⁻, or —C(O)O[CH₂]—N⁺CH₃(Et)₂X⁻.

In one embodiment, R⁷ and R¹⁰ in Formula V are both —C(O)O[CH₂]—N⁺(Et)₃X⁻. In yet another embodiment, R⁷ and R¹⁰ in Formula V are both —C(O)O[CH₂]₄—N(CH₃)₃X⁺.

For certain Formula IV or Formula V compounds R⁷ or R¹⁰, is a group selected from —C(O)NH[CH₂]NH₂, —C(O)NH[CH₂]₄NH₂, —C(O)NH[CH₂]NH(CH₃), —C(O)NH[CH₂]NH(formyl), or a PEG-containing prodrug such as —C(O)OCH₂—[OCH₂CH₂]₂—OCH₃, or —C(O)OCH₂—[OCH₂CH₂]₃—OCH₃ and R⁹ is propyl or pentyl.

According to one embodiment, R₇ and R₁₀ are each independently selected from —C(O)NH[CH₂]NH₂, —C(O)NH[CH₂]₄NH₂, —C(O)NH[CH₂]NH(CH₃), —C(O)NH[CH₂]NH(formyl), —C(O)OCH₂—[OCH₂CH₂]₂—OCH₃, and —C(O)OCH₂—[OCH₂CH₂]₃—OCH₃.

The prodrug of a cannabinoid or a cannabinoid analog according to Formula IV or Formula V may be purified prior to use. Purification is effected by procedures routinely used in the chemical and biochemical art, including solvent extraction or chromatographic purification methods. The purity of the purified prodrug product can be determined by thin layer chromatography (TLC), High Performance Liquid Chromatography coupled to a mass spectrometer (HPLC-MS), or by any suitable analytical technique. Nuclear magnetic resonance spectroscopy, mass spectral analysis, or UV, visible spectroscopy, are examples of analytical methods that can be used to confirm the identity of the inventive prodrugs.

Typically, the enantiomeric purity of the inventive prodrugs is from about 90% ee to about 100% ee, for instance, a prodrug of a cannabinoid or a cannabinoid analog according to the present invention can have an enantiomeric purity of about 91% ee, about 92% ee, about 93% ee, about 94% ee, about 95% ee, about 96% ee, about 97% ee, about 98% ee and about 99% ee. Cannabinoids exert different physiological properties and are known to lessen pain, stimulate appetite and have been tested as candidate therapeutics for treating a variety of disease conditions such as allergies, inflammation, infection, epilepsy, depression, migraine, bipolar disorders, anxiety disorder, and glaucoma. The physiological effects exerted by cannabinoids is affected by their ability to stimulate or deactivate the cannabinoid receptors, for instance the CB1, CB2 and CB3 receptors.

### Large Scale Production of a Cannabinoid Prodrug Using a Bioreactor

The present invention provides a system comprising a bioreactor for the large scale production of a cannabinoid prodrug. The bioreactor used for synthesizing a cannabinoid prodrug can be configured for batch synthesis or continuous synthesis so as to permit commercial production of pharmaceutically useful cannabinoid prodrugs.

In one embodiment, the system for producing a cannabinoid prodrug according to Formula VII or Formula VIII:

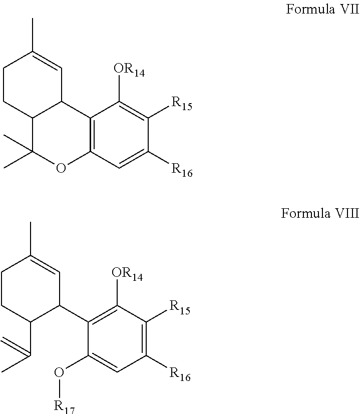

comprising:

(i) a bioreactor containing a reactant according to Formula VI, a solvent, and a cannabinoid synthase;

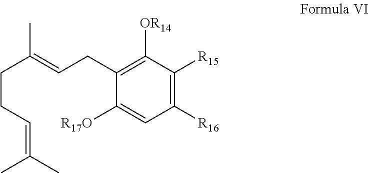

(ii) a control mechanism configured to control at least one condition of the bioreactor, wherein the compound according to Formula VI interacts with the cannabinoid synthase to produce a compound according to Formula VII or Formula VIII; and

(iii) optionally decarboxylating the Formula VII or Formula VIII compound.

For compounds according to Formula VI, VII, and VIII substituents R¹⁴ and R¹⁷ are each independently selected from the group consisting of —H, acetyl, propionyl, 3-hydroxy-2-methylpropionyl, TMS, TBDMS, benzyl, tetrahydropyran, —C(O)[CH₂]ₓ—C(O)OH, —C(O)[CH₂]ₓ—OR¹⁸, —C(O)[CHR¹⁸]ₓ—C(O)OH, —C(O)[CHR¹⁸]ₓ—OR¹⁹, —C(O)[CR¹⁸R¹⁹]ₓ—OR²⁰, —C(O)O[CH₂]ₓ—OR¹⁸, —C(O)—CH₂—[OCH₂CH₂]ₓ—OR¹⁸, —C(O)—C(O)—[OCH₂CH₂]ₓ—OR¹⁸, —C(O)[CH₂]ₓ—NR¹⁸R¹⁹, —C(O)O[CH₂]ₓ—NR¹⁸R¹⁹, —C(O)—NH—[CH₂]ₓ—NR¹⁸R¹⁹, —C(O)[CH₂]ₓ—N⁺(R¹⁸)(R¹⁹))(R²⁰)X⁻, —C(O)O[CH₂]ₓ—N⁺(R¹⁸)(R¹⁹))R²⁰)X⁻, —C(O)—NH—[CH₂]ₓ—N⁺(R¹⁸)(R¹⁹))(R²⁰)X⁻, a L-amino acid residue, a D-amino acid residue, a ß-amino acid residue, a γ-amino acid residue, —P(O)[OY](OZ), and —P(O)[NR¹⁸NR¹⁹][OY](OZ).

In one embodiment, R¹⁴ is —C(O)[CHR¹⁸]ₓ—OR¹⁹, —C(O)O[CH₂]ₓ—OR¹⁸, or —C(O)—CH₂—[OCH₂CH₂]ₓ—OR¹⁸, and substituents R¹⁸, and R¹⁹ are each independently —H, methyl, ethyl, or propyl.

According to another embodiment, when R¹⁴ is —C(O)[CHR¹⁸]ₓ—OR¹⁹, substituent R¹⁸ is —OH, —NH₂, —NH(CH₃), —NH(CH₂CH₃), N(CH₃)₂, —NH[C(O)H], —NH[C(O)CH₃], methyl, or ethyl and R¹⁹ is —H or methyl.

For certain Formula VII compounds, R¹⁴ is —C(O)O[CH₂]—OH, —C(O)O[CH₂]₂—OCH₃, —C(O)O[CH₂—CH(OH)]—OH, or —C(O)O[CH₂—CH(OH)]—OCH₃ and R¹⁷ is —H.

In one embodiment, substituents R¹⁴ and R¹⁷ are both —C(O)—CH₂—[OCH₂CH₂]₂—OH, or —C(O)—CH₂—[OCH₂CH₂]₃—OH. According to another embodiment, R¹⁴ is —C(O)—CH₂—[OCH₂CH₂]₂—OH, or —C(O)—CH₂—[OCH₂CH₂]₃—OH and R¹⁷ is —H.

In one embodiment, R¹⁴ is —C(O)[CH₂]ₓ—NR¹⁸R¹⁹, —C(O)O[CH₂]ₓ—NR¹⁸R¹⁹, —C(O)—NH—[CH₂]ₓ—NR¹⁸R¹⁹, or a quaternary ammonium group such as a group selected from —C(O)[CH₂]ₓ—N⁺(R¹⁸)(R¹⁹))(R²⁰)X⁻, —C(O)O[CH₂]ₓ—N⁺(R¹⁸)(R¹⁹))(R²⁰)X⁻, —C(O)—NH—[CH₂]ₓ—N⁺(R¹⁸)(R¹⁹))(R²⁰)X⁻.

For such Formula VII and VIII prodrugs, R¹⁸, R¹⁹, and R²⁰ are each independently selected from the group consisting of —H, —OH, formyl, acetyl, pivaloyl, methyl, ethyl, propyl, butyl, and pentyl and X⁻ is selected from chloride, acetate, malonate, or succinate. Subscripts “x” and “n” are independently selected from the group consisting of 0, 1, 2, 3, 4, 5, and 6.

In one embodiment, R¹⁴ is —C(O)—NH—[CH₂]₄—NH₂, —C(O)—NH—[CH₂]₄—NH(CH₃), or —C(O)—NH—[CH₂]₄—N(CH₃)₂ and R¹⁵ is —H.

According to another embodiment, R¹⁴ is —C(O)O[CH₂]—NH₂, —C(O)O[CH₂]—NH(CH₃), or —C(O)O[CH₂]—N(CH₃)₂ and R¹⁵ is —H.

In yet another embodiment, R¹⁴ is —C(O)[CH₂]—NH₃X—, —C(O)[CH₂]₂—N⁺H₃X⁻, —C(O)[CH₂]—N⁺H₂(CH₃)X⁻, or —C(O)[CH₂]—N⁺H(CH₃)₂X⁻, R¹⁵ is —H.

In yet another embodiment, R¹⁴ is —C(O)O[CH₂]—N⁺H₃X⁻, —C(O)O[CH₂]₂—N⁺H₃X⁻, —C(O)O[CH₂]—N⁺H₂(CH₃)X⁻, or —C(O)O[CH₂]—N⁺H(CH₃)₂X⁻, and R¹⁵ is —H.

In yet another embodiment, R¹⁴ is —C(O)NH[CH₂]—N⁺H₃X⁻, —C(O)NH[CH₂]₂—N⁺H₃X⁻, —C(O)NH[CH₂]—N⁺H₂(CH₃)X⁻, or —C(O)NH[CH₂]—N⁺H(CH₃)₂X⁻, and R¹⁵ is —H.

The present invention in one of its embodiments provides Formula VII compounds where R¹⁴ and R¹⁷ are both selected from the group consisting of —C(O)O[CH₂]—NH₂, —C(O)O[CH₂]—NH(CH₃), —C(O)O[CH₂]—N(CH₃)₂, —C(O)[CH₂]—N⁺H₃X⁻, —C(O)[CH₂]₂—N⁺H₃X⁻, —C(O)[CH₂]—N⁺H₂(CH₃)X⁻, —C(O)[CH₂]—N⁺H(CH₃)₂X⁻, —C(O)O[CH₂]—NH₃X—, —C(O)O[CH₂]₂—N⁺H₃X⁻, —C(O)O[CH₂]—N⁺H₂(CH₃)X⁻, —C(O)O[CH₂]—NH⁺(CH₃)₂X⁻, —C(O)NH[CH₂]—N⁺H₃X⁻, —C(O)NH[CH₂]₂—N⁺H₃X⁻, —C(O)NH[CH₂]—N⁺H₂(CH₃)X⁻, and —C(O)NH[CH₂]—N⁺H(CH₃)₂X⁻. Variable X⁻ is a counter ion and is an alkali metal cation, alkaline earth metal cation, or a counterion provided by a pharmaceutically acceptable acid.

In one embodiment, R¹⁵ is —COOH or —(CH₂)ₙCOOH and “n” is 1. According to another embodiment, the compound according to Formula VII or Formula VIII is de-carboxylated prior to pharmaceutical use and for such compounds R¹⁵ is —H.

In one embodiment, R¹⁵ is —COOR^{a}, for example —COOMe or —COOEt. For such compounds, hydrolysis of the ester by contact with a base such as a solution of sodium bicarbonate can occur prior to de-carboxylation.

R¹⁶ in Formula VI, VII and VIII is a group selected from (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene. In one embodiment, R¹⁶ is (C₁-C₁₀)alkyl, for example, methyl, ethyl, propyl, butyl, or pentyl.

In one embodiment the prodrug is —P(O)[OY](OZ), a phosphate selected from the group consisting of dihydrogen phosphate, alkali metal phosphate, alkaline earth metal phosphate, and the phosphate salt of an organic base.

According to this embodiment when the prodrug is a phosphate salt of an organic base, the organic base is selected from the group consisting of choline, betaine, caffeine, N,N-dibenzylethylenediamine, diethylamine, 2-di ethylaminoethanol, 2-dimethylaminoethanol, ethanolamine, ethylenediamine, N-ethylmorpholine, N-ethylpiperidine, glucamine, isopropylamine, methylglucamine, morpholine, piperidine, triethylamine, trimethylamine, tripropylamine, tetramethylammonium hydroxide, piperazine, histidine, arginine and lysine.

For certain Formula VII and VIII compounds, variables “Y” and “Z” are independently selected from the group consisting of —H, —H, (C₁-C₅)alkyl, alkali metal cations, alkaline earth metal cations, ammonium cation, and methyl ammonium cation.

In one embodiment, the system for producing a cannabinoid prodrug comprises a bioreactor that is configured for batch synthesis. Thus, the composition of the medium, concentration of the enzyme and substrate are fixed at the beginning of the bioenzymatic process and not allowed to change during catalysis. Synthesis is terminated when the concentration of the desired product in the medium of the bioreactor reaches a predetermined value or the concentration of substrate falls below a predetermined level, such as to a level where there is no detectable catalytic conversion of substrate to product.

In one embodiment, the cannabinoid acid synthase is His-tagged so as to facilitate separation of the enzyme from the product in the reaction medium by sequestering the His-tagged enzyme onto a nickel containing resin support within the bioreactor.

An alternative to the batch process mode is the continuous process mode in which a defined amount of substrate and medium are continuously added to the bioreactor while an equal amount of medium containing the cannabinoid product is simultaneously removed from the bioreactor to maintain a constant rate for formation of product.

The conditions of the bioreactor can be controlled using any control mechanism. The control mechanism may be coupled to the bioreactor or, alternatively, may interact with the bioreactor wirelessly or remotely. The control mechanism is used to control the conditions such the oxygen level, agitation, pH, and flow of materials (e.g. by controlling at least one pump) into and out of the bioreactor. In some embodiments, the control mechanism is configured to control the conditions of the bioreactor based on information obtained from an optical monitoring system.

The control mechanism may include a processing circuit having a processor and memory device configured to complete or facilitate various processes and functions, such as controlling the pH, temperature, and pressure in the bioreactor, or altering the flow rate of medium into or out of the bioreactor. Such control is affected by communicating with at least one sensor more than one sensor.

### Pharmaceutical Compositions

The prodrugs of Formula II or Formula III synthesized using the inventive method, or prodrugs according to Formula IV or V, or prodrugs according to Formula VII or Formula VIII produced using a bioreactor of the inventive system are administered to a patient or subject in need of treatment either alone or in combination with other compounds having similar or different biological activities. For example, the prodrugs and composition comprising the prodrugs of the invention can be administered in a combination therapy, i.e., either simultaneously in single or separate dosage forms or in separate dosage forms within hours or days of each other. Examples of such combination therapies include administering a composition comprising a prodrug according Formula II, III, IV, V, VII, and VIII with other pharmaceutical agents used to treat glaucoma, AIDS wasting, neuropathic pain, treatment of spasticity associated with multiple sclerosis, fibromyalgia and chemotherapy-induced nausea, emesis, wasting syndrome, HIV-wasting, alcohol use disorders, dystonia, multiple sclerosis, inflammatory bowel disorders, arthritis, dermatitis, Rheumatoid arthritis, systemic lupus erythematosus, anti-inflammatory, anti-convulsant, anti-psychotic, antioxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritic, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, osteoarthritis, rheumatoid arthritis, synovitis, juvenile rheumatoid arthritis and inhibition of hair growth.

The invention also provides a pharmaceutical composition comprising a pharmaceutically acceptable salt, solvate, or stereoisomer of a prodrug according to invention in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

The inventive compositions can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions in accordance with the invention include without limitation tablets, troches, lozenges, aqueous or oily suspensions, dispersible powders or granules, emulsion, hard or soft capsules, syrups or elixirs.

Encompassed within the scope of the invention are pharmaceutical compositions suitable for single unit dosages that comprise a prodrug of the invention its pharmaceutically acceptable stereoisomer, salt, solvate, hydrate, or tautomer and a pharmaceutically acceptable carrier.

Inventive compositions suitable for oral use may be prepared according to any method known to the art for the manufacture of pharmaceutical compositions. For instance, liquid formulations of the inventive prodrugs contain one or more agents selected from the group consisting of sweetening agents, flavoring agents, coloring agents and preserving agents in order to provide pharmaceutically elegant and palatable preparations of the inventive prodrug.

For tablet compositions, the active ingredient in admixture with non-toxic pharmaceutically acceptable excipients is used for the manufacture of tablets. Exemplary of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, the inventive prodrug is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally occurring phosphatide, for example, lecithin, polyoxyethylene stearate, heptadecaethyleneoxycetanol, polyoxyethylene sorbitol monooleate, polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the prodrug in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, or an aqueous suspension. This suspension may be formulated according to the known art using those suitable dispersing or wetting agents and suspending agents. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

The total amount by weight of a cannabinoid prodrug of the invention in a pharmaceutical composition is from about 0.1% to about 95%. By way of illustration, the amount of a cannabinoid prodrug by weight of the pharmaceutical composition, such as a cannabidiol prodrug, a THC prodrug, or a THC-v prodrug of the invention can be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%6, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95%.

In one embodiment, the pharmaceutical composition comprises a total amount by weight of a cannabinoid prodrug, of about 1% to about 10%; about 2% to about 10%; about 3% to about 10%; about 4% to about 10%; about 5% to about 10%; about 6% to about 10%; about 7% to about 10%; about 8% to about 10%; about 9% to about 10%; about 1% to about 9%; about 2% to about 9%; about 3% to about 9%; about 4% to about 9%; about 5% to about 9%; about 6% to about 9%; about 7% to about 9%; about 8% to about 9%; about 1% to about 8%; about 2% to about 8%; about 3% to about 8%; about 4% to about 8%; about 5%, to about 8%; about 6% to about 8%; about 7% about 8%; about 1% to about 7%; about 2% to about 7%; about 3%, to about 7%; about 4% to about 7%; about 5% to about 7%; about 6% to about 7%; about 1% to about 6%; about 2% to about 6%; about 3% to about 6%; about 4% to about 6%; about 5% to about 6%; about 1% to about 5%; about 2% to about 5%; about 3% to about 5%; about 4% to about 5%; about 1% to about 4%; about 2% to about 4%; about 3% to about 4%; about 1% to about 3%; about 2% to about 3%; or about 1% to about 2%.

### EXAMPLES

### A. Chemical Synthesis

### A. Synthesis of Olivetol

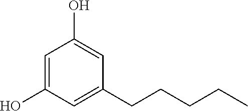

Olivetol was synthesized using a published procedure (Focella, A, et al., J. Org. Chem., Vol. 42, No. 21, (1977), p. 3456-3457).

### i. Methyl 6-N-Pentyl-2-hydroxy-4-oxo-cyclohex-2-ene-1-carboxylate

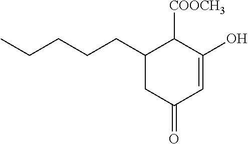 [SMILES: CCCCCC1CC(=O)C=C(O)C1C(=O)OC]

To a stirring solution of sodium methoxide (32.4 g, 0.60 mol) and dimethyl malonate (90 g, 0.68 mol) in 230 mL of anhydrous methanol was added portion wise 75 g (0.48 mol) of 90% 3-nonen-2-one. The reaction mixture was then refluxed for 3 h under N₂ and allowed to cool to room temperature. The solvent was distilled under reduced pressure and the residue dissolved in 350 mL of water. The slurry of white crystals and the almost clear solution was extracted thrice with 80 mL of chloroform. The aqueous layer was acidified to pH 4 with concentrated HCl and the white precipitate that formed was allowed to stand overnight prior to filtration. The crystals were dried at 50° C. under high vacuum for 5 hours to yield 106.5 g (0.4416 mol) (92%) of methyl 6-n-Pentyl-2-hydroxy-4-oxo-cyclohex-2-ene-1-carboxylate (mp 96-98 C). The product was recrystallized using a mixture of petroleum ether:ethyl acetate (9:1), and gave 94 g of pure methyl 6-n-Pentyl-2-hydroxy-4-oxo-cyclohex-2-ene-1-carboxylate (melting point of 98-100 C).

### ii. 1-n-Pentyl-3,5-dihydroxybenzene (Olivetol)

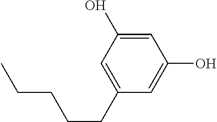

To a stirring ice-cooled solution of methyl 6-N-pentyl-2-hydroxy-4-oxo-cyclohex-2-ene-1-carboxylate (58.4 g, 0.24 mol) dissolved in 115 mL dimethylformamide was added dropwise 37.9 g (0.23 mol) of bromine dissolved in 60 mL of dimethylformamide. At the end of the addition (ca. 90 min) the reaction mixture was slowly heated to 80° C. during which time the evolution of carbon dioxide became quite vigorous.

The reaction was maintained at this temperature until gas evolution had ceased following which the reaction was further heated to 160° C. and held at this temperature for approximately 10 hours. After heating, the reaction was allowed to cool and the solvent DMF was removed under reduced pressure. The residue thus obtained was treated with water (80 mL) and extracted twice with 250 mL of ether. The combined ether layers were washed with water, then washed with 2×80 mL of a 100% solution of sodium bisulfite, 2×80 mL of a 100% solution of acetic acid, and then again with water.

After drying over anhydrous sodium sulfate the solvent was removed under reduced pressure to give 46.8 g of viscous oil. The oil was distilled under reduced pressure to give 30.3 g (0.168 mol) (69.3%) of olivetol as product. HPLC analysis indicated 97.5%0 purity.

### B. Synthesis of CBG

CBG was synthesized following the protocol disclosed by Taura et al., (1996), The Journal of Biological Chemistry, Vol. 271, No. 21, p. 17411-17416.

### Synthesis of 2-[(2E)-3,7-dimethylocta-2,6-dienyl]-5-pentyl-benzene-1,3-diol (Cannabigerol (CBG))

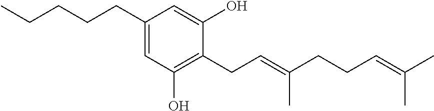 [SMILES: CCCCCc1cc(O)c(C/C=C(\C)CCC=C(C)C)c(O)c1]

Geraniol (3 g, 0.0194 mol) and olivetol (2 g, 0.0111 mol) were dissolved in 400 mL of chloroform containing 80 mg of p-toluenesulfonic acid as catalyst and the reaction mixture was stirred at room temperature for 12 h in the dark. After 12 hours, the reaction mixture was washed with saturated sodium bicarbonate (400 mL) and then with H₂O (400 mL). The chloroform layer was concentrated at 40° C. under reduced pressure, and the residue obtained was chromatographed on a 2.0 cm×25 cm silica gel column using benzene (1000 mL) as the eluent to give 1.4 g (0.00442 mol×39.9%) CBG as product.

Alternatively crude CBG was purified as follows. To a 250 mL beaker was added 7.25 g crude CBG and 50 mL benzene. The flask was swirled to dissolve the CBG and 50 g silica gel was added, along with a stir bar. The solution was stirred overnight, and then poured into a 44 cm×2.75 cm column. The column was eluted with 300 mL benzene. The eluent, approximately 70 mL fractions were assayed for CBG. Fractions 1, 2, and 3 (˜230 mL) that contained CBG were combined and the solvent removed under pressure to give 6.464 g residue containing >80% CBG, having a purity suitable for use in the next synthetic step.

In one embodiment, crude CBG was purified by mixing 7.25 g crude CBG residue with a slurry of silica gel (50 mL), in a 250 ml Beaker. This mixture was slowly agitated for 1 hour and then vacuum filtered using a fine mesh filter paper. The filter cake was washed with 250 ml benzene until a clear filtrate was obtained. The solvent from the filtrate was removed under reduced pressure to give 6.567 g of a residue having >80% CBG.

### C. Synthesis of Methylmagnesium Carbonate (MMC)

Methylmagnesium Carbonate (MMC) was synthesized following the protocol disclosed by Balasubrahmanyam et al., (1973), Organic Synthesis, Collective Volume V, John Wiley & Sons, Inc., p. 439-444.

A dry 2 L, three necked flask was fitted with a mechanical stirrer, a condenser, and a 1 L, pressure-equalizing addition funnel, the top of which was fitted with a gas inlet tube. A clean, dry magnesium ribbon (40.0 g, 1.65 mol) was placed in the flask and the system was flushed with nitrogen prior to the addition of anhydrous methanol (600 mL). Hydrogen gas evolution was controlled by cooling the reaction mixture. When evolution of hydrogen gas ceased, a slow stream of nitrogen was passed through the system and the condenser replaced by a total condensation-partial take-off distillation head. The nitrogen flow was stopped and the bulk of the methanol distilled from the solution under reduced pressure. Distillation was stopped when stirring of the pasty suspension of magnesium methoxide was no longer practical. The system was again flushed using nitrogen and the outlet from the distillation head was attached to a small trap containing mineral oil so that the volume of gas escaping from the reaction system could be estimated.

Anhydrous dimethylformamide (DMF)(700 mL) was added to the reaction flask, and the resulting suspension was stirred vigorously while a stream of anhydrous carbon dioxide was passed into the reaction vessel through the gas inlet tube attached to the addition funnel. The dissolution of carbon dioxide was accompanied by an exothermic reaction with the suspended magnesium methoxide. When no more CO₂ is absorbed, the colorless solution was heated under a slow stream of CO₂ gas until the temperature of the liquid distilling reached 140° C., indicating that residual methanol had been removed from the reaction mixture. The reaction mixture was flushed using a slow stream of nitrogen to aid in cooling the mixture to room temperature under an inert atmosphere. This yielded a solution having 536 mg MMC/mL of DMF.⁸

### D. Synthesis of CBGA (3-[3,7-dimethyl-2,6-octadiene]-2,4-dihydroxy-6-pentyl benzene-1-carboxylic acid)

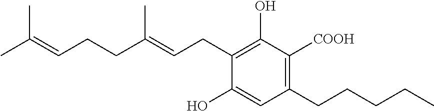 [SMILES: CCCCCc1cc(O)c(C/C=C(\C)CCC=C(C)C)c(O)c1C(=O)O]

6-carboxylic acid-2-[(2E)-3,7-dimethylocta-2,6-dienyl]-5-pentyl-benzene-1,3-diol, Cannabigerolic Acid (CBGA) was prepared as follows. To a 10 mL conical flask was added 1 mL of a DMF solution of MMC. To this solution was added 2-[(2E)-3,7-dimethylocta-2,6-dienyl]-5-pentyl-benzene-1,3-diol (120 mg, 0.379 mmol). The flask was heated at 120° C. for 1 hour, following which the reaction mixture was dissolved in 100 mL of chloroform:methanol (2:1) solution. The pH of this solution was adjusted with dilute HCl to pH 2.0, and then partitioned using 50 mL H₂O.

The organic layer was dried over sodium sulfate and the solvent was removed by evaporation. HPLC analysis of the crude reaction showed ˜40% conversion of CBG to CBGA.

Alternatively, 3.16 g (10 mmols) of CBG (or any other neutral cannabinoid), 8.63 g (100 mmols) magnesium methylate and 44 g (1 mol) of dry ice were sealed in a pressure compatible vessel. The vessel is heated to 50° C., and the temperature held at this value for three hours. Following heating, the vessel is cooled to room temperature and slowly vented. The reaction mixture was dissolved in 100 mL of a chloroform:methanol (2:1) solvent. The pH of this solution was adjusted with dilute HCl to pH 2.0 and this solution was then partitioned using 50 mL of H₂O. The organic layer was dried over sodium sulfate and the solvent was removed by evaporation. HPLC analysis of crude reaction mixture showed ˜85% conversion of CBG to CBGA using this protocol.

Crude CBGA was purified by chromatography using a 2.0 cm×25 cm silica gel column. The product was eluted using a mixture of n-hexane:ethyl acetate (2:1) (1000 mL), to obtain 45 mg (0.125 mmol)(37.5%) of the desired product.

Alternatively, ultra high purity CBGA was obtained by chromatographing the crude using LH-20 lipophilic resin as the medium. 400 g of LH-20 Sephadex resin was first swollen using 2 L of DCM:chloroform (4:1) solvent. The swollen resin was gravity packed in a 44×2.75 cm column. The column was loaded with 2.1 g of crude CBGA dissolved in a minimum amount of DCM:chloroform (4:1) solvent and eluted with 1.7 L of the same solvent. 100 mL fractions were collected. The unreacted CBG was eluted as a yellow/orange solution using this solvent system. After the passage of about 1.7 L of this solvent, no more yellow/orange fraction were observed and the eluting solvent was changed to 100% acetone to elute the bound CBGA.

The fractions containing CBGA were pooled and the solvent was removed to obtain 0.52 g CBGA (˜90% recovery). Increasing the volume of DCM:chloroform (4:1)solvent passed through the column prior to eluting with acetone, yielded CBGA having purity greater than 99.5%.

### E. Synthesis of TBDMS-CBGA (3-[3,7-dimethylocta-2,6-diene]-2-hydroxy-6-pentyl-4-[t-butyldimethylsilyloxy]benzoic acid) or TBDMS-CBGA-ethyl ester (Ethyl-3-[3,7-dimethylocta-2,6-diene]-2-hydroxy-6-pentyl-4-[t-butyldimethylsilyloxy]benzoate)

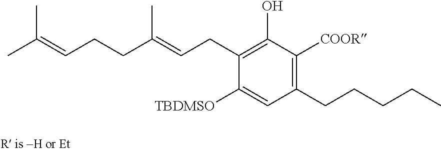

To a cold stirring solution of CBGA or CBGA-ethyl ester in DCM under an atmosphere of argon is added t-butyldimethylsilyl chloride (1.0 eq.) and imidazole. TLC is used to monitor reaction progress. The reaction is quenched upon completion by the addition of brine. The organic layer was separated and dried using anhydrous magnesium sulfate prior to purification and use. If CBGA-ethyl ester is used as the starting material, the product can be hydrolyzed to the corresponding acid, if necessary, prior to enzyme-catalyzed synthesis of the cannabinoid prodrug.

A similar protocol is used for synthesizing 3-[3,7-dimethylocta-2,6-diene]-2-hydroxy-6-pentyl-4-[trimethylsilyloxy]benzoic acid via the reaction of CBGA or CBGA-ester with trimethylsilyl chloride in the presence of a base such as imidazole.

### B. Synthesis of Formula I Compounds

### a. Synthesis of Cannabigerolic Acid 3,6,9,12-tetraoxatridecanoyl ester

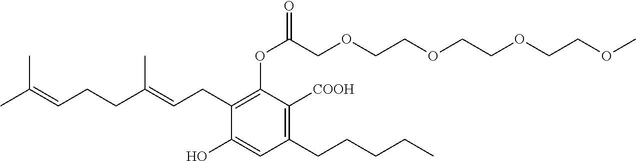 [SMILES: CCCCCc1cc(O)c(C/C=C(\C)CCC=C(C)C)c(OC(=O)COCCOCCOCCOC)c1C(=O)O]

4-dimethylaminopyridine (DMAP) is added to a solution of 3,6,9,12-tetraoxatridecanoic acid in dichloromethane (DCM). To this solution, add N,N′-dicyclohexylcarbodiimide or carbonyldiimidazole. After stirring at room temperature, add a DCM solution of TBDMS-CBGA or TBDMS-CBGA-ethyl ester dropwise. The reaction mixture is stirred at room temperature overnight, filtered and the filtrate is concentrated under reduced pressure prior purification of the crude product by silica gel column chromatography.

The TBDMS protecting group is removed by adding tetrabutylammonium fluoride or triethylamine trihydrofluoride to a DCM solution of cannabigerolic acid 3,6,9,12-tetraoxatridecanoyl ester at −15° C. The reaction mixture is stirred at this temperature and TLC is used to monitor progress of deprotection. Following de-protection ethyl acetate (EtOAc is added to the reaction and the organic layer extracted (λ3) using a dilute aqueous solution of sodium bicarbonate.

The combined organic layers are dried and the solvent evaporated under reduced pressure prior to purification.

### b. Synthesis of Cannabigerolic Acid N,N-dimethylglycyl ester

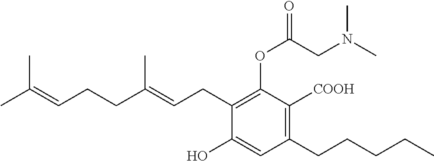 [SMILES: CCCCCc1cc(O)c(C/C=C(\C)CCC=C(C)C)c(OC(=O)CN(C)C)c1C(=O)O]

4-dimethylaminopyridine (DMAP) is added to a DCM solution of N,N-dimethyl glycine. To this solution, add N,N′-dicyclohexylcarbodiimide. After stirring at room temperature, add a DCM solution of TBDMS-CBGA or TBDMS-CBGA-ethyl ester dropwise. Continue stirring the reaction mixture at room temperature overnight. The next day, the reaction mixture is filtered, and the filtrate is concentrated under reduced pressure prior purification of the crude product by silica gel column chromatography.

De-protection of the TBDMS protecting group is carried out using protocols described herein.

### c Synthesis of Cannabigerolic Acid (R)-2,3-dihydroxypropyl carbonate

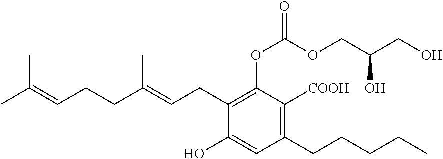 [SMILES: CCCCCc1cc(O)c(C/C=C(\C)CCC=C(C)C)c(OC(=O)OC[C@H](O)CO)c1C(=O)O]

Accordingly, triethylamine is added to a solution of (S)-2,3-bis(t-butyldimethylsilyloxy)propan-1-ol in dichloromethane under an Argon atmosphere at 0° C. To this solution is added triphosgene and stirring of the resultant reaction mixture is continued at 0 C for approximately 3-5 hours. The resultant solution of (S)-2,3-bis(t-butyldimethylsilyloxy)propyl chloroformate is then cannulated to a stirring DCM solution of TBDMS-CBGA or TBDMS-CBGA-ethyl ester and triethylamine at 0° C. that is maintained under an inert atmosphere of Argon.

The resultant mixture is then stirred at room temperature and the reaction progress monitored periodically by TLC. Following completion, the reaction mixture is diluted, filtered, and the filtrate concentrated under reduced pressure to obtain CBGA (S)-2,3-bis(t-butyldimethylsilyloxy)propyl carbonate as an oil.

Removal of the TBDMS protecting groups is achieved by dissolving the crude product in cold DCM at −15 C. This cold DCM solution is then contacted with a cold solution of triethylamine trihydrofluoride (2N), and stirred at 5° C. for 65 h. Following stirring EtOAc is added to the resultant mixture followed by the addition of a dilute aqueous solution of sodium bicarbonate at 0° C. and vigorous stirring. The organic layers containing the descried crude are combined and dried prior to purification using HPLC or silica gel column chromatography.

Synthetic protocols described above are used to produce other inventive cannabinoid prodrugs, for example, the cannabinoid prodrugs illustrated in Tables 1 and 2 above. It is understood that the above synthetic protocols can be modified to accommodate chemical and reactivity differences of moieties used to manufacture the inventive prodrugs. However, such modifications of the synthetic protocol are well within the purview of a person of ordinary skill in the chemical art.

### C. Prodrug Synthesis

An illustrative protocol for monitoring the enzyme-catalyzed formation of an inventive prodrug is as follows. Enzyme-catalyzed synthesis of the inventive prodrugs is conducted in a 1.5 ml Eppendorf snap cap tube. 25 μl of the substrate, for example a Formula I compound dissolved in DMSO at 1.0 mg/ml is added to 200 μl of 100 mM citrate buffer, pH 4.85. This solution is incubated at 30° C. for 2 hours with 25 μl of a cannabinoid synthase enzyme. The reaction is terminated by the addition of 250 μl MeOH and analyzed by HPLC. Enzyme activity is tested under a variety of conditions as follows:

1. Different solvents and mixtures of solvents as described above are tested to enhance substrate solubility and improve reaction rate.
2. Assays will be run at pH's 4, 5, 6, 7, and 8.
3. Enzyme assays are run in either Sodium phosphate buffer or Citrate buffer with or without SDS or Triton-X. Some assays are run in a mixed solvent system that includes DMSO, DMF, IPA, or cyclodextrin (CD) at varying concentrations.
4. Bioenzymatic synthesis of a prodrug are monitored after incubating the reaction mixture for a time interval of 1 minute to about 4 days.

### Enzyme Catalyzed Synthesis of a Formula II or Formula III Compound.

2-hydroxypropyl-β-cyclodextrin (HPfβCD; Kleptose® HPB), sulfobutylether β-cyclodextrin sodium salt (SBEPCD; Captisol®), or a randomly methylated β-cyclodextrin (RMβCD; concentration 35 g/L) is added to a 10 mM sodium phosphate buffer (pH 5.0). The solution is stirred to form a homogenous solution prior to the addition of a Formula I compound. After mixing at room temperature for 1-2 min, a buffered solution of THCA synthase is added and the reaction mixture incubated at 30° C. At uniform intervals of time, aliquots (10 μl) of the reaction mixture are taken and added to an eppendorf tube containing ethanol (50 μl), to denature the enzyme. After centrifugation at 10,000 rpm for 5 minutes, the ethanol layer is separated from the denatured protein precipitate, transferred to a clean eppendorf tube and the solvent evaporated using a stream of nitrogen.

The residue thus obtained is reconstituted in buffer and the progress of the enzyme catalyzed formation of a Formula II or Formula III prodrug is quantitated by reverse-phase HPLC.

Alternatively, the reaction mixture is diluted 10:1 with 95% EtOH to cause cyclodextrin to precipitate out while leaving the prodrugs of the cannabinoid or cannabinoid analog as well as unreacted Formula I compound in solution. After removing the supernatant the solvent is evaporated and the residue thus obtained analyzed by HPLC after reconstitution in buffer.

The precipitate of cyclodextrin is washed with excess 90% EtOH, and dried to permit its reuse in a future reaction.

### 1. Synthesis of a Formula II Prodrug

Scheme 1 illustrates the bioenzymatic synthesis of a cannabinoid prodrug according to Formula II

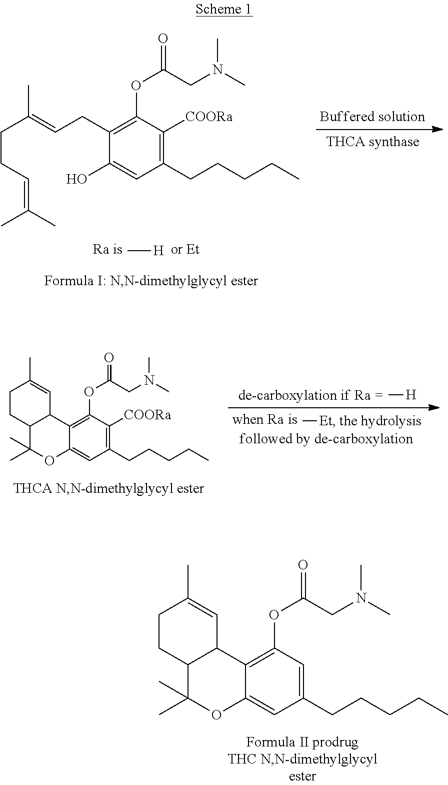

CBGA N,N-dimethylglycyl ester prepared using the protocol described above is added to a solution comprising cyclodextrin and buffer in a 1.0 ml eppendorf tube. After complete dissolution of the CBGA ester, the solution is incubated in a controlled temperature water bath maintained at 37° C., for at least 15 minutes before adding an known amount of a buffered solution of THCA synthase.

Following addition of the enzyme, a known aliquot of the reaction mixture, approximately 25 ul, is withdrawn at fixed intervals of time and the enzyme denatured by adding a fixed volume of ethanol. Following centrifugation of the precipitate, the ethanol layer is separated, dried and reconstituted in buffer. Progress of the reaction can be followed spectrophotometrically or using HPLC.

The product, THCA N,N-dimethylglycyl ester is separated from the reaction mixture by denaturing the enzyme using ethanol and evaporating the ethanol layer containing THCA N,N-dimethylglycyl ester to dryness.

The Formula II prodrug, THC N,N-dimethylglycyl ester is obtained in two ways: (1) De-carboxylation by heating the a buffered solution of THCA N,N-dimethylglycyl ester, or (2) directly contacting the ethanol solution of THCA N,N-dimethylglycyl ester that is obtained following denaturation of the enzyme.

Synthesis of a Formula II prodrug on a commercial scale occurs using a bioreactor that contains a buffered solution of the reactant CBGA N,N-dimethylglycyl ester in contact with a cannabinoid synthase. Reaction progress is monitored spectrophotometrically by removing aliquots of the reaction mixture. The enzyme is separated from the product, THCA N,N-dimethylglycyl ester by passing the reaction mixture over a Ni-bound column. Because the enzyme used for large-scale synthesis of prodrugs comprises a His-tag, the enzyme will bind to the Ni-column while the product and unreacted starting materials will remain in the eluent.

The desired product, THCA N,N-dimethylglycyl ester, is purified by extraction into an organic solvent or by HPLC. THCA N,N-dimethylglycyl ester is de-carboxylated by contacting a solution of THCA N,N-dimethylglycyl ester to heat.

### 2. Synthesis of a Formula III Prodrug

Schemes 2 and 3 respectively illustrate the bioenzymatic synthesis of a monoester and a diester prodrug of a cannabinoid according to Formula III. The protocol for the enzyme catalyzed conversion of CBGA N,N-dimethylglycyl ester, or CBGA bis(N,N-dimethylglycyl) ester to the corresponding CBD N,N-dimethylglycyl ester and CBD bis(N,N-dimethylglycyl) ester respectively is similar to the one described above for Formula II prodrugs.

The monoester prodrug can be chemically converted to a diester prodrug by contacting the monoester with N,N-dimethylglycylcarbonyl imidazole as described above or by any coupling protocol known to one of ordinary skill in the chemical art.

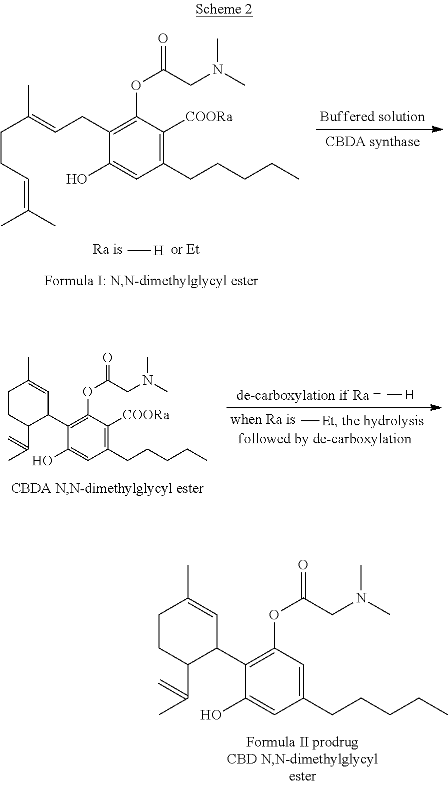

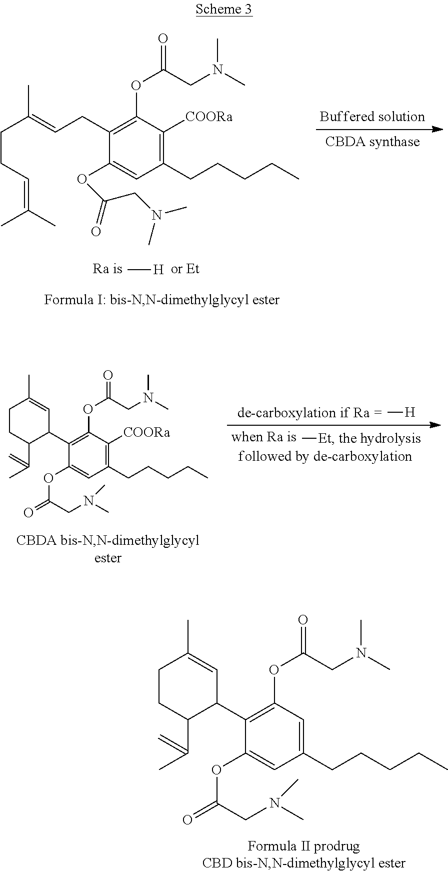

Large-scale synthesis of Formula III prodrugs is achieved in a bioreactor, using a method similar to the one described above for Formula II prodrugs.

### D. Purification of the Prodrugs

The cannabinoid prodrugs produced by bioenzymatic synthetic protocol described herein are purified by several analytical methods, including HPLC, size exclusion chromatography, and extraction into an organic solvent. The fractions corresponding to the desired prodrug product can be pooled and lyophilized to dryness.

### E. Methods of Use

The naturally occurring cannabinoid tetrahydrocannabinol (THC), is gaining acceptance as a therapeutic for treating a wide range of medical conditions, including glaucoma, AIDS wasting, neuropathic pain, treatment of spasticity associated with multiple sclerosis, fibromyalgia and chemotherapy-induced nausea. THC is also effective in the treatment of allergies, inflammation, infection, epilepsy, depression, migraine, bipolar disorders, anxiety disorder, drug dependency and drug withdrawal syndromes.

The present invention provides prodrugs of natural cannabinoids as therapeutics for treating the above mentioned disorders. For instance, the inventive prodrugs when formulated for parenteral delivery are candidate therapeutics for alleviating pain. Such treatment is effected by administering a pharmaceutically acceptable formulation of the inventive prodrug alone or in combination with another pharmaceutical agent with known activity for reducing pain. The two pharmaceutical agents can be administered together or separately and the dose of each pharmaceutical agent is determined by the prescribing physician.

Prodrugs in accordance with the invention are also candidate therapeutics for treating inflammation. For instance, the inventive prodrugs can be administered to alleviate inflammation of the joints and associated pain in a subject with rheumatoid arthritis. The inventive prodrugs can be administered alone or in conjunction with a COX-inhibitor if necessary, at doses suitable for such treatment and deemed necessary by the prescribing physician.

We claim:

1. A pharmaceutical composition comprising a cannabinoid prodrug according to Formula IV or Formula V, or a pharmaceutically acceptable salt thereof: 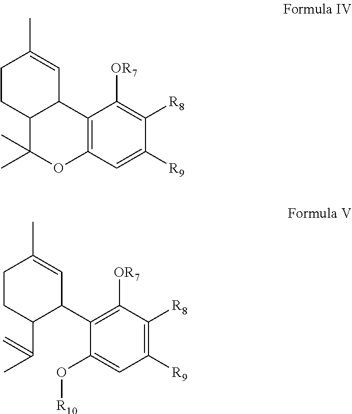
wherein
in Formula IV, R⁷ is selected from the group consisting of propionyl, 3-hydroxy-2-methylpropionyl, tetrahydropyranyl, —C(O)[CH₂]ₓ—C(O)OH, —C(O)[CH₂]ₓ—OR¹¹, —C(O)[CHR¹¹]ₓ—C(O)OH, —C(O)[CHR¹¹]ₓ—OR¹², —C(O)[CR¹¹R¹²]ₓ—OR¹³, —C(O)O[CH₂]ₓ—OR¹¹, —C(O)—CH₂—[OCH₂CH₂]ₓ—OR¹¹, —C(O)—C(O)—[OCH₂CH₂]ₓ—OR¹¹, —C(O)[CH₂]ₓ—NR¹¹R¹², —C(O)O[CH₂]ₓ—NR¹¹R¹², —C(O)—NH—[CH₂]ₓ—NR¹¹R¹², —C(O)[CH₂]ₓ—N⁺(R¹¹)(R¹²))(R¹³)X⁻, —C(O)O[CH₂]ₓ—N⁺(R¹¹)(R¹²))(R¹³)X⁻, —C(O)—NH—[CH₂]ₓ—N⁺(R¹¹)(R¹²))(R¹³)X⁻, a L-amino acid residue, a D-amino acid residue, a ß-amino acid residue, a γ-amino acid residue, —P(O)[OY](OZ), and —P(O)[NR¹¹R¹²][OY](OZ);
in Formula V, R⁷ and R¹⁰ are each independently selected from the group consisting of —H, propionyl, 3-hydroxy-2-methylpropionyl, tetrahydropyranyl, —C(O)[CH₂]ₓ—C(O)OH, —C(O)[CH₂]ₓ—OR¹¹, —C(O)[CHR¹¹]ₓ—C(O)OH, —C(O)[CHR¹¹]ₓ—OR¹², —C(O)[CR¹¹R¹²]ₓ—OR¹³, —C(O)O[CH₂]ₓ—OR¹¹, —C(O)—CH₂—[OCH₂CH₂]ₓ—OR¹¹, —C(O)—C(O)—[OCH₂CH₂]ₓ—OR¹¹, —C(O)[CH₂]ₓ—NR¹¹R¹², —C(O)O[CH₂]ₓ—NR¹¹R¹², —C(O)—NH—[CH₂]ₓ—NR¹¹R¹², —C(O)[CH₂]ₓ—N⁺(R¹¹)(R¹²))(R¹³)X⁻, —C(O)O[CH₂]ₓ—N⁺(R¹¹)(R¹²))R¹³)X⁻, —C(O)—NH—[CH₂]ₓ—N⁺(R¹¹)(R¹²))(R¹³)X⁻, a L-amino acid residue, a D-amino acid residue, a ß-amino acid residue, a γ-amino acid residue, —P(O)[OY](OZ), and —P(O)[NR¹¹NR¹²][OY](OZ),
wherein R⁷ and R¹⁰ are not simultaneously H;
R⁸ is —H, —COOH, —COOR^{a}, or —(CH₂)ₙCOOH;
R⁹ is selected from the group consisting of methyl, ethyl, propyl, isopropyl, butyl, sec-butyl, tert-butyl, hexyl, isohexyl, and neohexyl;
R¹¹, R¹² and R¹³ are each independently selected from the group consisting of —H, —OH, formyl, acetyl, pivaloyl, —NH₂, —NH(CH₃), —NH(CH₂CH₃), —N(CH₃)₂, —NH[C(O)H], —NH[C(O)CH₃], and (C₁-C₅)alkyl;
R^{a} is (C₁-C₁₀)alkyl;
“X” is a counter ion derived from a pharmaceutically acceptable acid;
“Y” and “Z” are each independently selected from the group consisting of —H, (C₁-C₅)alkyl, alkali metal cations, alkaline earth metal cations, ammonium cation, methyl ammonium cation, and pharmaceutically acceptable bases;
subscript “x” is selected from the group consisting of 1, 2, 3, 4, 5, and 6; and
subscript “n” is selected from the group consisting of 0, 1, 2, 3, 4, 5, and 6;
and a pharmaceutically acceptable carrier.

2. The pharmaceutical composition of claim 1, wherein R⁷ is selected from the group consisting of —C(O)[CH₂]ₓ—C(O)OH, —C(O)[CH₂]ₓ—OR¹¹, —C(O)[CH₂]ₓ—NR¹¹R¹², —C(O)—CH₂—[OCH₂CH₂]ₓ—OR¹¹, and —C(O)[CH₂]ₓ—N⁺(R¹¹)(R¹²)(R¹³)X⁻.

3. The pharmaceutical composition of claim 1, wherein R⁸ is —H or —COOH, and R⁹ is propyl or butyl.

4. The pharmaceutical composition of claim 1, wherein R⁸ is —H and R⁹ is propyl.

5. The pharmaceutical composition according to claim 1, wherein the cannabinoid prodrug is selected from the following table: 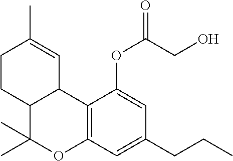 [SMILES: CCCc1cc(OC(=O)CO)c2c(c1)OC(C)(C)C1CCC(C)=CC21] 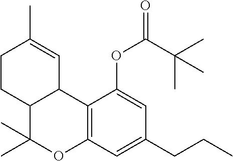 [SMILES: CCCc1cc(OC(=O)C(C)(C)C)c2c(c1)OC(C)(C)C1CCC(C)=CC21] 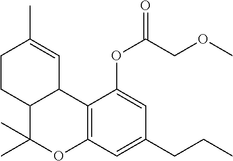 [SMILES: CCCc1cc(OC(=O)COC)c2c(c1)OC(C)(C)C1CCC(C)=CC21] 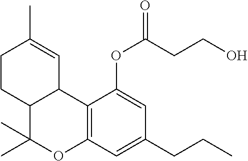 [SMILES: CCCc1cc(OC(=O)CCO)c2c(c1)OC(C)(C)C1CCC(C)=CC21] 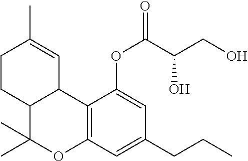 [SMILES: CCCc1cc(OC(=O)[C@@H](O)CO)c2c(c1)OC(C)(C)C1CCC(C)=CC21] 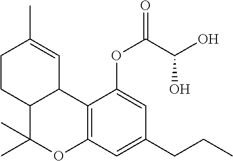 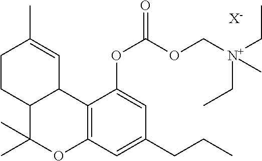 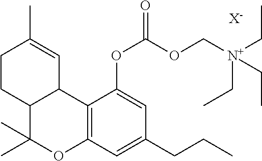 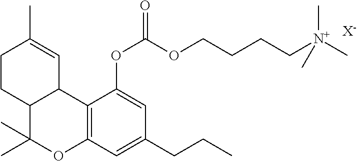 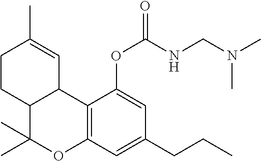 [SMILES: CCCc1cc(OC(=O)NCN(C)C)c2c(c1)OC(C)(C)C1CCC(C)=CC21] 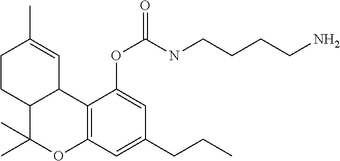 [SMILES: CCCc1cc(OC(=O)NCCCCN)c2c(c1)OC(C)(C)C1CCC(C)=CC21] 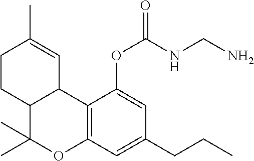 [SMILES: CCCc1cc(OC(=O)NCN)c2c(c1)OC(C)(C)C1CCC(C)=CC21] 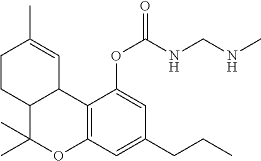 [SMILES: CCCc1cc(OC(=O)NCNC)c2c(c1)OC(C)(C)C1CCC(C)=CC21] 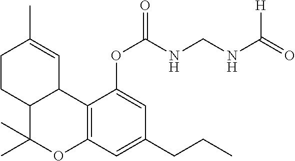 [SMILES: CCCc1cc(OC(=O)NCNC=O)c2c(c1)OC(C)(C)C1CCC(C)=CC21] 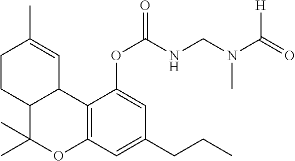 [SMILES: CCCc1cc(OC(=O)NCN(C)C=O)c2c(c1)OC(C)(C)C1CCC(C)=CC21] 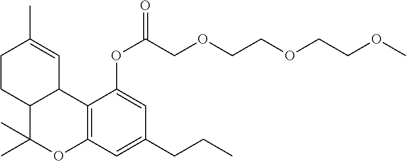 [SMILES: CCCc1cc(OC(=O)COCCOCCOC)c2c(c1)OC(C)(C)C1CCC(C)=CC21] 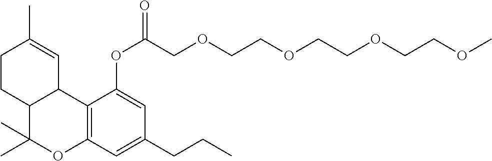 [SMILES: CCCc1cc(OC(=O)COCCOCCOCCOC)c2c(c1)OC(C)(C)C1CCC(C)=CC21] 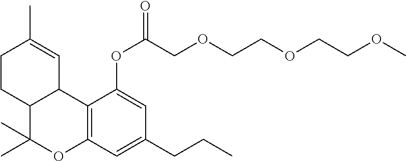 [SMILES: CCCc1cc(OC(=O)COCCOCCOC)c2c(c1)OC(C)(C)C1CCC(C)=CC21]
or a pharmaceutically acceptable salt thereof.

6. A pharmaceutical composition comprising a cannabinoid prodrug, or a pharmaceutically acceptable salt thereof, selected from the following table:

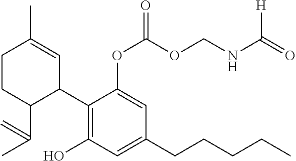 [SMILES: C=C(C)C1CCC(C)=CC1c1c(O)cc(CCCCC)cc1OC(=O)OCNC=O]

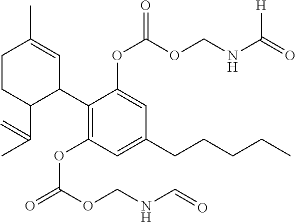 [SMILES: C=C(C)C1CCC(C)=CC1c1c(OC(=O)OCNC=O)cc(CCCCC)cc1OC(=O)OCNC=O]

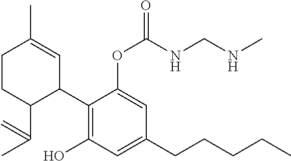 [SMILES: C=C(C)C1CCC(C)=CC1c1c(O)cc(CCCCC)cc1OC(=O)NCNC]

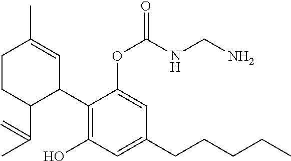 [SMILES: C=C(C)C1CCC(C)=CC1c1c(O)cc(CCCCC)cc1OC(=O)NCN]

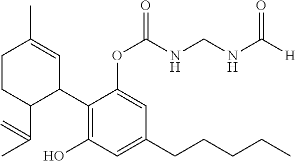 [SMILES: C=C(C)C1CCC(C)=CC1c1c(O)cc(CCCCC)cc1OC(=O)NCNC=O]

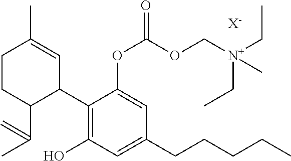

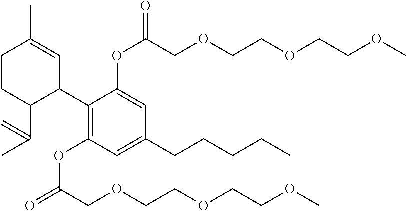 [SMILES: C=C(C)C1CCC(C)=CC1c1c(OC(=O)COCCOCCOC)cc(CCCCC)cc1OC(=O)COCCOCCOC]

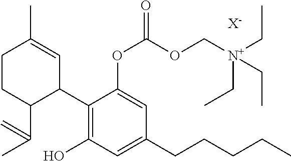

7. A method for producing a cannabinoid prodrug of Formula II or Formula III: 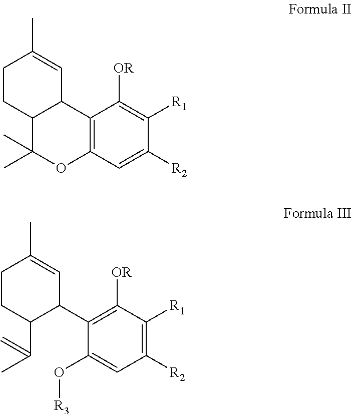
comprising
(i) contacting a compound according to Formula I in a mixture comprising an aqueous buffer and at least one solvent selected from the group consisting of dimethylsulfoxide, dimethyl formamide, and iso-propyl alcohol: 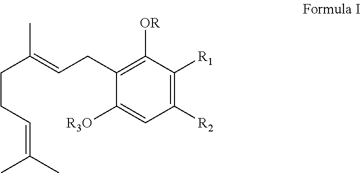
with a cannabinoid synthase to produce a compound according to Formula II or Formula III; and
(ii) optionally decarboxylating the Formula II or Formula III compound;
wherein
R and R³ are each independently selected from the group consisting of —H, acetyl, propionyl, 3-hydroxy-2-methylpropionyl, TMS, TBDMS, benzyl, tetrahydropyran, —C(O)[CH₂]ₓ—C(O)OH, —C(O)[CH₂]ₓ—OR⁴, —C(O)[CHR₄]ₓ—C(O)OH, —C(O)[CHR⁴]ₓ—OR⁵, —C(O)[CR⁴R⁵]ₓ—OR⁶, —C(O)O[CH₂]ₓ—OR⁴, —C(O)—CH₂—[OCH₂CH₂]ₓ—OR⁴, —C(O)—C(O)—[OCH₂CH₂]ₓ—OR⁴, —C(O)[CH₂]ₓ—NR⁴R⁵, —C(O)O[CH₂]ₓ—NR⁴R⁵, —C(O)—NH—[CH₂]ₓ—NR⁴R⁵, —C(O)[CH₂]ₓ—N⁺(R⁴)(R⁵))(R⁶)X⁻, —C(O)O[CH₂]ₓ—N⁺(R⁴)R⁵)(R⁶)X⁻, —C(O)—NH—[CH₂]ₓ—N⁺(R⁴)(R⁵))R⁶)X⁻, a L-amino acid residue, a D-amino acid residue, a ß-amino acid residue, a γ-amino acid residue, —P(O)[OY](OZ), and —P(O)[NR⁴NR⁵][OY](OZ);
R¹ is —H, —COOH, —COOR^{a}, or —(CH₂)COOH;
R² is selected from the group consisting of (C₁-C₁₀)alkyl, (C₂-C₁₀)alkenyl, (C₂-C₁₀)alkynyl, (C₃-C₁₀)cycloalkyl, (C₃-C₁₀)cycloalkylalkylene, (C₃-C₁₀)aryl, and (C₃-C₁₀)arylalkylene;
R⁴, R⁵, and R⁶ are each independently selected from the group consisting of —H, —OH, formyl, acetyl, pivaloyl, —NH₂, —NH(CH₃), —NH(CH₂CH₃), N(CH₃)₂, —NH[C(O)H], —NH[C(O)CH₃], and (C₁-C₅)alkyl;
R^{a} is (C₁-C₁₀)alkyl;
“X” is a counter ion derived from a pharmaceutically acceptable acid;
“Y” and “Z” are each independently selected from the group consisting of —H, (C₁-C₅)alkyl, alkali metal cations, alkaline earth metal cations, ammonium cation, methyl ammonium cation, and pharmaceutically acceptable bases; and
subscripts “x” and “n” are independently selected from the group consisting of 0, 1, 2, 3, 4, 5, and 6.

8. The method according to claim 7, wherein R¹ is —COOH, and R² is (C₁-C₁₀)alkyl.

9. The method according to claim 8, wherein R² is propyl or pentyl.

10. The method according to claim 8, wherein R is selected from the group consisting of —C(O)[CH₂]ₓ—C(O)OH, —C(O)[CH₂]ₓ—OR⁴, —C(O)[CH₂]ₓ—NR⁴R⁵, and —C(O)—CH₂—[OCH₂CH₂]ₓ—OR⁴.

11. The method according to claim 10, wherein R is —C(O)[CH₂]ₓ—OR⁴, subscript “x” is 1, 2, 3, or 4, and R⁴ is —H, or (C₁-C₅)alkyl.

12. The method according to claim 10, wherein R is —C(O)—CH₂—[OCH₂CH₂]ₓ—OR⁴, R⁴ is methyl, and subscript “x” is 1, 2, 3, or 4.

13. The method according to claim 10, wherein R is —C(O)[CH₂]ₓ—NR⁴R⁵ and subscript “x” is 1, 2, 3, or 4.

14. The method according to claim 13, wherein R⁴ and R⁵ are each independently —H, or (C₁-C₅)alkyl.

15. The method according to claim 7, wherein the organic solvent is dimethylsulfoxide.

16. The method according to claim 7, wherein the aqueous buffer is selected from the group consisting of citrate buffer, phosphate buffer, HEPES, Tris buffer, MOPS, and glycine buffer.

17. The method according to claim 7, wherein the concentration of the organic solvent is between 10% and 50% (v/v).

18. The method according to claim 17, wherein the concentration of the organic solvent is between 10/o and 300/0 (v/v).

19. The method according to claim 18, wherein the concentration of the organic solvent is between 10% and 20% (v/v).
